(19) United States
(12) Patent Application Publication
(10) Pub. No.: US 2014/0336248 A1
(43) Pub. Date: Nov. 13, 2014

(54) THERAPEUTIC COMPOSITIONS AND RELATED METHODS OF USE

(71) Applicant: VERASTEM, INC., Cambridge, MA (US)

(72) Inventors: Wendi Ma, Shanghai (CN); Peter Elliott, Marlborough, MA (US); Mahesh Padval, Needham, MA (US)

(21) Appl. No.: 14/362,879

(22) PCT Filed: Dec. 5, 2012
(86) PCT No.: PCT/US12/67952

§ 371 Date: Jun. 4, 2014

Related U.S. Application Data
(60) Provisional application No. 61/584,589, filed on Jan. 9, 2012.

(30) Foreign Application Priority Data
Dec. 5, 2011 (CN) 201110405324.X

(51) Int. Cl. A61K 31/35; A61K 45/06

(52) U.S. Cl. CPC A61K 31/35; A61K 45/06
USPC 514/460; 435/375

(57) ABSTRACT

Aqueous compositions comprising salinomycin, or a pharmaceutically acceptable salt thereof, miscible organic solvents, and solubilizers and/or emulsifying agents are disclosed. The formulations disclosed herein are useful in the treatment of cancer, especially cancers associated with cancer stem cells or cancerous mesenchymal cells. The method of treating a subject may further comprise identifying a subject having a disorder suitable for treatment with the aqueous compositions described, comprising detecting one or more biomarkers predictive of the prevalence of a cancer having or enriched for cancer stem cells (CSCs).

This application claims priority to Chinese Application Number 201110405324.X, filed Dec. 5, 2011, and U.S. Provisional Application Ser. No. 61/584,589, filed Jan. 9, 2012, the disclosures of each of which are considered part of (and is incorporated by reference in) the disclosure of this application.

### BACKGROUND OF INVENTION

Salinomycin is an ionophore, having the following structure:

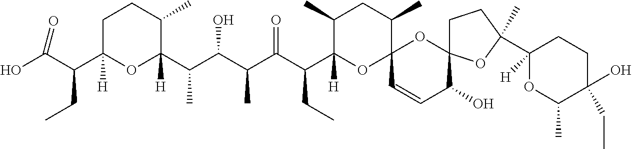 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C]

It has been used for its antibacterial properties in the treatment of coccidiosis (a parasitic disease of the intestinal tract) in domesticated animals, e.g., dogs, cats, cattle, horses, swine, and poultry. Some studies have indicated that salinomycin may also be useful in the treatment of certain cancers.

### SUMMARY OF INVENTION

Described herein are aqueous compositions, e.g., pharmaceutical compositions, comprising salinomycin, or a salt thereof. The aqueous compositions can be used, for example, in the treatment of a disorder such as cancer or a microbial infection. In some embodiments the aqueous compositions are used for the treatment of cancer, e.g., the aqueous compositions can be administered to a subject to kill, inhibit the growth of, limit the proliferation of, or cause other beneficial changes to a subject (e.g., a human) having cancer cells (e.g., a tumor). Methods of treating a subject using these aqueous compositions, e.g., treating a subject identified as having cancer, are described.

Exemplary cancers include those cancers comprising a solid tumor and cancers of the blood. In some embodiments, the cancer is, or is characterized as comprising or enriched for, cancer stem cells (CSCs), tumor-initiating cells, mesenchymal cells, or mesenchymal-like cells associated with cancer, or mesenchymal cancerous cells. In some embodiments, the cancer has metastasized, or the cancer is characterized as having or being enriched for metastatic cells, or the cancer has a likelihood of metastasizing.

In some embodiments, the method of treating a subject may further comprise identifying a subject having a disorder suitable for treatment with an aqueous composition described herein. For example, a subject having been identified as having cancer. Exemplary cancers include those described herein, including those characterized as comprising or enriched for, cancer stem cells (CSCs), tumor-initiating cells, mesenchymal cells, or mesenchymal-like cells associated with cancer, or mesenchymal cancerous cells. In some embodiments, the aqueous compositions are administered to a subject that has been identified with a biomarker described herein (e.g., a biomarker predictive of the prevalence of a cancer having or enriched for, cancer stem cells (CSCs), tumor-initiating cells, mesenchymal cells, or mesenchymal-like cells associated with cancer, or mesenchymal cancerous cells). In some embodiments, the subject has been identified with a plurality of biomarkers described herein, e.g., wherein the plurality of biomarkers, when evaluated together are predictive of the prevalence of a cancer having or enriched for, cancer stem cells (CSCs), tumor-initiating cells, mesenchymal cells, or mesenchymal-like cells associated with cancer, or mesenchymal cancerous cells. In some embodiments, an aqueous composition described herein is administered to a subject having a disorder, wherein the subject (or a sample from the subject) has been determined to be sensitive to salinomycin (e.g., wherein treatment of the subject with salinomycin would be beneficial).

In some embodiments, a method of treating a subject with an aqueous composition described herein may additionally comprise administering an additional cancer therapy (e.g., surgery, radiation, chemotherapy, hormonal therapy, vaccines, antibodies, gene therapy or other targeted therapies). For example, a subject may be treated with a combination of an aqueous composition described herein and another cancer therapy. The composition and the additional cancer therapy be administered simultaneously or sequentially, e.g., the composition may be administered before or after the additional cancer therapy.

In some embodiments the aqueous compositions are used for the treatment of microbial infections. For example, the aqueous compositions can be administered to a subject to kill, inhibit the growth of, limit the proliferation of, or cause other beneficial changes to a subject (e.g., a human) infected with microbes, e.g., bacteria, fungi, or protists, e.g., coccidia. Methods of treating a subject using these aqueous compositions, e.g., treating a subject as having an undesired microbial infection, are described.

In one aspect, the invention features an aqueous composition, e.g., a pharmaceutical composition, comprising salinomycin, or a salt of salinomycin, e.g., a sodium, ammonium, lithium, cesium, or potassium salt, e.g., salinomycin sodium. In some embodiments, the aqueous composition additionally comprises a solubilizer and/or emulsifying agent. In some embodiments, the aqueous composition additionally comprises a miscible organic solvent. In some embodiments, the aqueous composition additionally comprises a preservative. In some embodiments, the aqueous composition comprises a solubilizer and/or emulsifying agent, and a miscible organic solvent, for delivering salinomycin, or a salt thereof, to a subject at from about 0.5 to about 1.5 mg/kg intravenously.

In one embodiment, the aqueous composition, e.g., the pharmaceutical composition, comprises about 5% of miscible organic solvent and about 5% of emulsifying agent, the balance being water. In an embodiment, the aqueous composition comprises from about 0.01 to 25 mg/ml salinomycin. In one embodiment, the aqueous composition comprises from about 0.1 to 20 mg/ml salinomycin. In one embodiment, the composition comprises from about 1 to about 15 mg/ml salinomycin. In one embodiment, the aqueous composition comprises from about 8 to about 12 mg/ml.

In one aspect, the invention features a dosage form comprising an aqueous composition, e.g., a pharmaceutical composition, of salinomycin or a salinomycin salt thereof, e.g., salinomycin sodium, for example as described herein. In some embodiments, the dosage form is a parenteral dosage form, e.g., for administration to a subject intravenously.

In one aspect, the invention features a kit comprising an aqueous composition, e.g., a pharmaceutical composition, of salinomycin or a salinomycin salt thereof, e.g., salinomycin sodium, for example as described herein.

In one aspect, the invention features an aqueous composition, e.g., a pharmaceutical composition, comprising from about 0.01 to about 15 mg/mL salinomycin from about 0.5 to about 5% (v/v) ethanol, from about 1 to about 10% (v/v) propylene glycol, and from about 1 to about 20% (v/v) Solutol® HS 15. In an embodiment, the aqueous composition comprises from about 0.01 to 25 mg/ml salinomycin. In one embodiment, the aqueous composition comprises from about 0.1 to 20 mg/ml salinomycin. In one embodiment, the composition comprises from about 1 to about 15 mg/ml salinomycin. In one embodiment, the aqueous composition comprises from about 8 to about 12 mg/ml.

In some embodiments, the composition comprises from about 2 to about 8 mg/mL of salinomycin or a salt thereof, such as salinomycin sodium (e.g., about 3 to about 7 mg/mL or about 5 mg/mL of salinomycin or a salt thereof such as salinomycin sodium). In some embodiments, the composition comprises 2 to about 8 mg/mL of salinomycin or a salt thereof, such as salinomycin sodium (e.g., about 3 to about 7 mg/mL or about 5 mg/mL of salinomycin or a salt thereof such as salinomycin sodium), and an excipient. Exemplary excipients include solutol (e.g., solutol HS15), propylene glycol, ethanol and water.

In some embodiments, the composition comprises from about 2 to about 8 mg/mL of salinomycin sodium (e.g., about 3 to about 7 mg/mL or about 5 mg/mL of salinomycin sodium at pre-dilution concentrations). In some embodiments, the composition comprises 5 mg/mL of salinomycin sodium at pre-dilution concentrations. In some embodiments, the composition comprises 5 mg/mL of salinomycin sodium at a pre-dilution concentration, solutol HS15, propylene glycol, ethanol and water.

In some embodiments, the composition comprises salinomycin sodium and a diluent, e.g., normal saline, 5% dextrose injection, and 5% dextrose injection in saline. In some embodiments, the composition comprises from about 2 to about 8 mg/mL of salinomycin or a salt thereof, such as salinomycin sodium (e.g., about 3 to about 7 mg/mL or about 5 mg/mL of salinomycin sodium at pre-dilution concentrations), and a diluent. In some embodiments, the composition comprises 2 to about 8 mg/mL of salinomycin sodium (e.g., about 3 to about 7 mg/mL or about 5 mg/mL of salinomycin sodium at pre-dilution concentrations), and a diluent. In some embodiments, the concentration of salinomycin sodium is 5 mg/mL and the diluent is normal saline. In some embodiments, the concentration of salinomycin sodium is 5 mg/mL (at a pre-dilution concentration) and the diluent is 5% dextrose injection. In some embodiments, the concentration of salinomycin sodium is 5 mg/mL (at a pre-dilution concentration) and the diluent is 5% dextrose injection in saline.

In some embodiments, the composition comprises from about 0.05 mg/mL to about 0.5 mg/mL of salinomycin sodium (e.g., about 0.07 to about 0.3 mg/mL or about 0.1 mg/mL of salinomycin sodium at a post-dilution concentration). In some embodiments, the composition comprises 0.1 mg/mL of salinomycin sodium at a post-dilution concentration, solutol HS15, propylene glycol, ethanol and water. For example, in some embodiments, 10 mL of the composition comprising salinomycin, or a salt thereof, e.g., salinomycin sodium at a pre-dilution concentration of 5 mg/mL is diluted with 500 mL of diluent.

In some embodiments, the composition comprises from about 0.1 mg/mL to about 1.0 mg/mL of salinomycin sodium (e.g., about 0.2 to about 0.8 mg/mL or about 0.5 mg/mL of salinomycin sodium at a post-dilution concentration). In some embodiments, the composition comprises 0.5 mg/mL of salinomycin sodium, at a post-dilution concentration, solutol HS15, propylene glycol, ethanol and water. For example, in some embodiments, 10 mL of the composition comprising salinomycin, or a salt thereof, e.g., salinomycin sodium, at a pre-dilution concentration of 5 mg/mL is diluted with 100 mL of diluent.

In another aspect, the invention features a composition comprising salinomycin or a salt thereof, e.g., a formulation that provides chemical and physical stability. For example, the formulation can slowdown or prevent physical changes to the active pharmaceutical ingredient (API), e.g., salinomycin or a salt thereof. Examples of physical changes include, but are not limited to, appearance, precipitation, clarity and color of the formulation solution, crystal modification, e.g., polymorphism, and particle size. In another embodiment, the formulation can slowdown or prevent chemical changes, such as, for example, an increase in degradation or a decrease of assay.

In some embodiments, salinomycin or salt thereof in the composition, e.g., formulation, retains its chemical integrity and potency. In some embodiments, the original physical properties, including appearance, palatability, uniformity, dissolution, and suspendability are retained.

In one aspect, the invention features a method of treating a subject, the method comprising administering to a subject an aqueous composition, e.g., a pharmaceutical composition, described herein. In some embodiments, the subject has a proliferative disease.

In an embodiment, the proliferative disease is cancer. In some embodiments, the cancer is breast cancer. In some embodiments, the breast cancer does not express genes for estrogen receptors, progesterone receptors, or Her2/neu receptors, e.g., triple-negative breast cancer.

In some embodiments, the method further comprises administering an additional cancer treatment e.g., surgery, radiation therapy, chemotherapy, or targeted therapy, in combination with the aqueous composition. In some embodiments, the additional cancer treatment is administered simultaneously with the aqueous composition. In some embodiments, the additional cancer treatment is administered sequentially with the aqueous composition. In some embodiments the additional cancer treatment is chemotherapy. In some embodiments, the chemotherapy is a taxane (e.g., docitaxel, paclitaxel, or cabazitaxel). In some embodiments, the chemotherapy is a platinum compound, e.g., cisplatin. In some embodiments, the chemotherapy is a PARP inhibitor, e.g., inaparib. In some embodiments, the chemotherapy is an anthracycline, e.g., doxorubicin.

In some embodiments the subject is administered the aqueous composition parenterally, e.g., subcutaneously, or intravenously. In some embodiments, the aqueous compositions are dosed intravenously at a dose of from about 0.001 to about 10 mg/kg, e.g., 0.005 to 5 mg/kg, e.g., 0.01 to 1 mg/kg, e.g., 0.1 to 1 mg/kg, e.g., 0.1, or 0.2, or 0.3, or 0.4, or 0.5, or 0.6, or 0.7, or 0.8, or 0.9, or 1.0 mg/kg. In some embodiments, the subject is administered the aqueous composition orally as a liquid. In some embodiments, the aqueous compositions are dosed orally at a dose of 0.01 to 100 mg/kg, e.g., 0.05 to 50 mg/kg, e.g., 0.1 to 40 mg/kg, e.g., 10 to 30 mg/kg, e.g., about 10, 11, 12, 13, 14, 15, 16, 17, 18, 19, 20, 21, 22, 23, 24, 25, 26, 27, 28, or 29, or 30 mg/kg.

In one aspect, the invention features a method of treating a subject, the method comprising selecting a subject having a cancer in which a cancer stem cell or mesenchymal cancer cell biomarker has been detected, and administering to the subject an aqueous salinomycin composition, e.g., a pharmaceutical composition, described herein. In some embodiments, the cancer is breast cancer. In some embodiments, the breast cancer does not express genes for estrogen receptors, progesterone receptors, or Her2/neu receptors, e.g., triple-negative breast cancer.

In one aspect, the invention features a method of inhibiting the proliferation of cancer stem cells or mesenchymal cancerous cells, the method comprising contacting the cancer stem cells or mesenchymal cancerous cells with an aqueous composition e.g., a pharmaceutical composition, of salinomycin or a salinomycin salt, e.g., salinomycin sodium. In some embodiment, the method comprises selectively inhibiting cancer stem cells or mesenchymal cancerous cells relative to cancer cells that are not cancer stem cells and/or relative to non-cancer cells and/or relative to cancer cells that are not mesenchymal, e.g., epithelial cells.

In one aspect, the invention features a method of making an aqueous salinomycin composition, e.g., a pharmaceutical composition, the method comprising contacting salinomycin or a salt thereof with a miscible organic solvent to make a solution, contacting the resulting organic solution with a solubilizer and/or emulsifying agent to form a second solution, and contacting the second solution with water, to thereby make an aqueous salinomycin composition.

### BRIEF DESCRIPTION OF THE DRAWINGS

FIG. 1 depicts the weight of mice administered intravenous aqueous compositions of salinomycin.

FIG. 2 depicts the weight of mice administered intravenous aqueous compositions of salinomycin.

FIG. 3 depicts the weight of mice administered oral aqueous compositions of salinomycin.

FIG. 4 depicts the weight of mice administered intravenous aqueous compositions of salinomycin.

FIG. 5 depicts the weight of mice administered intravenous aqueous compositions of salinomycin.

FIG. 6 depicts the weight of mice administered intravenous aqueous compositions of salinomycin.

FIG. 7 depicts the weight of mice administered intravenous aqueous compositions of salinomycin.

FIG. 8 depicts the weight of mice administered intravenous aqueous compositions of salinomycin.

FIG. 9 depicts the weight of mice administered intravenous aqueous compositions of salinomycin.

FIG. 10 depicts the weight of mice administered intravenous aqueous compositions of salinomycin.

FIG. 11 depicts a comparative profile of the X-Ray Powder Diffraction (XRPD) analysis for Active Pharmaceutical Ingredient (API), formulation and crystals obtained at 50° C. after 1 week.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting.

### DEFINITIONS

The term “pharmaceutically acceptable carrier or adjuvant” refers to a carrier or adjuvant that may be administered to a subject, together with a compound of this invention, and which does not destroy the pharmacological activity thereof and is nontoxic when administered in doses sufficient to deliver a therapeutic amount of the compound.

As used herein, the term “aqueous composition” is defined as a composition comprising water. The water may be pure, e.g., deionized, millipure, or distilled water. The water may be a buffered water solution or another pharmaceutically acceptable aqueous composition, for example, a water solution containing pharmaceutically acceptable salts. The pH of the buffered water solution may be from pH=3 to pH=11, typically pH=5 to pH=9, more typically a pH of about 7.

As used herein, the term “treat” or “treatment” is defined as the application or administration of a compound, alone or in combination with, a second compound to a subject, e.g., a subject, or application or administration of the compound to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to minimize at least one symptom of the disorder or to delay onset of at least one symptom of the disorder).

As used herein, a cancer that is enriched with a type of cell, e.g., cancer stem cells, e.g., mesenchymal cells, e.g., tumor-initiating cells, e.g., mesenchymal-like cells associated with cancer, or mesenchymal cancerous cells, indicates a cancer that has more (e.g., 50% more, 2 times more, 5 times more, 10 times more, 100 time more) of a type of cell, e.g., cancer stem cells, e.g., mesenchymal cells, e.g., tumor-initiating cells, e.g., mesenchymal-like cells associated with cancer, or mesenchymal cancerous cells, than a benign tumor, benign growth, or a nonproliferative disorder. The extent of this enrichment can be determined with known screening tests for identifying these cells, e.g., cancer stem cells, e.g., mesenchymal cells, e.g., tumor-initiating cells, e.g., mesenchymal-like cells associated with cancer, or mesenchymal cancerous cells, in a sample of the cancer.

As used herein, a cancer that has a likelihood of metastasizing is a cancer that is known to metastasize in greater than 1%, e.g, greater than 5%, e.g, greater than 10%, e.g, greater than 15%, e.g, greater than 20%, e.g, greater than 25%, e.g, greater than 30%, e.g, greater than 35%, e.g, greater than 40%, e.g, greater than 45%, e.g, greater than 50%, e.g, greater than 90%, of the subjects who have historically presented with the disorder. Such cancers include, e.g., lung cancer, breast cancer, melanoma, colon cancer, pancreatic cancer, and cervical cancer. A cancer that has a likelihood of metastasizing may also refer to a cancer for which a cell or tissue sample has tested positive for the presence of a cancer stem cell, or a mesenchymal cell, or a tumor-initiating cell, or a mesenchymal-like cell associated with cancer, or a mesenchymal cancerous cell. A cancer that has a likelihood of metastasizing may also refer to a cancer for which a cell or tissue sample has tested positive for at least one biomarker, e.g., a plurality of biomarkers, associated with a cancer stem cell, or a mesenchymal cell, or a tumor-initiating cell, or a mesenchymal-like cell associated with cancer, or a mesenchymal cancerous cell or at least one biomarker, e.g., a plurality of biomarkers, indicative of Wnt pathway activation.

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, “a prophylactically effective amount” of the compound refers to an amount effective, upon single- or multiple-dose administration to the subject, in reducing at least one symptom of the disorder or to delay onset of at least one symptom of the disorder.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention (for example an imidate ester of an amide), which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

As used herein, the term “subject” is intended to include human and non-human animals. Exemplary human subjects include a human subject having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

### Compositions and Methods of Making Compositions

Described herein are aqueous compositions, e.g., pharmaceutical compositions, comprising salinomycin, or a salt of salinomycin, e.g., salinomycin sodium. In some embodiments, the aqueous composition additionally comprises a solubilizer and/or emulsifying agent. In some embodiments, the aqueous composition additionally comprises a miscible organic solvent. In some embodiments, the aqueous composition additionally comprises a preservative. In some embodiments, the aqueous composition comprises salinomycin, a solubilizer and/or emulsifying agent, a miscible organic solvent, and optionally a preservative. In one embodiment, the aqueous composition comprises about 5% of miscible organic solvent (v/v) and about 5% of solubilizer and/or emulsifying agent (v/v), the balance being water. In an embodiment, the aqueous composition comprises from about 0.01 to 25 mg/ml salinomycin. In one embodiment, the aqueous composition comprises from about 0.1 to 20 mg/ml salinomycin. In one embodiment, the composition comprises from about 1 to about 15 mg/ml salinomycin. In one embodiment, the aqueous composition comprises from about 8 to about 12 mg/ml.

As described above, the aqueous compositions, e.g., pharmaceutical compositions, comprise salinomycin. In some embodiments, the aqueous composition comprises a salt of salinomycin. Exemplary salinomycin salts include any pharmaceutically acceptable salt. Exemplary pharmaceutically acceptable salts include e.g., a salt derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. In some embodiments, the salt is a sodium salt such as sodium salinomycin or salinomycin sodium.

In some embodiments the aqueous composition, e.g., pharmaceutical compositions, comprises salinomycin salt hydrates, e.g., salinomycin sodium hydrates. In some embodiments, the salinomycin, salinomycin salts, or salinomycin salt hydrates are enantiomerically enriched, e.g., comprising at least 90%, e.g., at least 95%, e.g., at least 99%, e.g., at least 99.9% of a salinomycin salt having specific chiral centers, e.g., the structure shown below

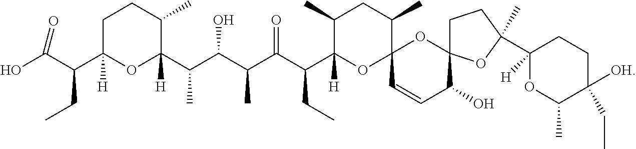 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C]

In some embodiments the aqueous composition comprises an analog of salinomycin or a pharmaceutically-acceptable salt of an analog of salinomycin.

In some embodiments the salinomycin composition comprises at least 0.01 mg/ml salinomycin, e.g., at least 0.05 mg/ml, at least 0.1 mg/ml, at least 0.5 mg/ml, at least 1.0 mg/ml, at least 2.0 mg/ml, at least 3.0 mg/ml, at least 5.0 mg/ml, at least 10 mg/ml, at least 15 mg/ml, at least 20 mg/ml, at least 25 mg/ml, at least 30 mg/ml, at least 40 mg/ml, or at least 50 mg/ml salinomycin. In an embodiment, the aqueous composition comprises from about 0.01 to 25 mg/ml salinomycin. In one embodiment, the aqueous composition comprises from about 0.1 to 20 mg/ml salinomycin. In one embodiment, the composition comprises from about 1 to about 15 mg/ml salinomycin. In one embodiment, the aqueous composition comprises from about 8 to about 12 mg/ml.

In some embodiments, the aqueous composition additionally comprises a solubilizer and/or emulsifying agent. Exemplary solubilizers and/or emulsifying agents include amphiphilic molecules such as a long-chain amphiphilic molecules. In some embodiments, the amphiphilic molecule is non-ionic. In some embodiment, the solubilizer and/or emulsifying agent comprises a polyalkylene oxide such as PEG. In some embodiments, the solubilizer and/or emulsifying agent is a polysorbate, e.g., a polyoxyethylene derivative of sorbitan monolaurate, e.g., a Tween such as Tween® 80. In some embodiments, the solubilizers and/or emulsifying agents are mixtures of polyethylene glycol and derivatives of hydroxystearate, e.g., mono- and di-esters of 12-hydroxystearic acid, e.g., a solutol such as Solutol® HS 15. In some embodiments, the solubilizers and/or emulsifying agents are polyethoxylated castor oil, e.g., a Cremophor such as Cremophor EL. Other solubilizers and/or emulsifying agents that have been recognized as safe by an appropriate regulatory body, e.g., the U.S. Food and Drug Administration (FDA), may also be used. In some embodiments, the salinomycin composition comprises at least 0.1% solubilizer and/or emulsifying agent (v/v), e.g., at least 0.5%, at least 1%, at least 5%, at least 10%, at least 15%, at least 20%, at least 25%, at least 30% solubilizer and/or emulsifying agent (v/v).

In some embodiments, the aqueous composition additionally comprises a miscible organic solvent, e.g., an alcohol, an organic acid, or a polar-organic solvent. In some embodiments, the miscible organic solvent is an alcohol e.g., ethanol or propylene glycol. In some embodiments, the miscible organic solvent is an organic acid, e.g., propanoic acid. In some embodiments, the miscible organic solvent is a polar-organic solvent or polar aprotic solvent, e.g., DMSO. In some embodiments, the salinomycin composition comprises at least 1% miscible organic solvent (v/v), e.g., at least 5%, at least 10%, at least 15%, at least 20%, at least 25%, at least 30%, at least 35%, at least 40% miscible organic solvent (v/v).

In some embodiments, the aqueous composition (e.g., an aqueous composition) described herein comprises a stabilizer. Exemplary stabilizers include chelating agents, for example, EDTA or EDTA salts, e.g., disodium EDTA, or citric acid. Exemplary stabilizers also include antioxidants, such as ascorbic acid, tocopherol/tocopherol derivatives, and metabisulfites, e.g., sodium metabisulfite, as well as preservatives, such as benzyl alcohol, parabens, or cholorobutanol.

In addition to the components described above, the aqueous compositions described herein (e.g., aqueous compositions) can include additional ingredients such as additional pharmaceutically acceptable carriers, adjuvants and vehicles. Exemplary pharmaceutically acceptable carriers, adjuvants and vehicles include ion exchangers, lecithin, self-emulsifying drug delivery systems (SEDDS) such as d-alpha-tocopherol polyethyleneglycol 1000 succinate, emulsifying agents used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and polyethylene glycol. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The aqueous compositions, e.g. pharmaceutical compositions, described herein can be made using a variety of techniques. Generally, an amount of a salinomycin salt, e.g., salinomycin sodium is contacted with (e.g., mixed with) a miscible organic solvent or a combination of miscible organic solvents to make a first solution. The first solution is then contacted with an emulsifying agent (solubilizer) to make a second solution. The second solution is typically mixed, for example with stirring, shaking, or sonication, until a clear solution is formed. Once the second solution has been mixed, the second solution is diluted with water to achieve the desired concentration. One of skill in the art would recognize that the steps described above need not be completed in the disclosed order, for example the salinomycin salt and miscible organic solvent and the solubilizer and/or emulsifying agent could be mixed together initially. Other variations would be obvious to one of skill in the art and are intended to be captured by this disclosure.

While it is generally possible to create compositions comprising salinomycin or salinomycin salts using the techniques described herein, some compositions described herein are well suited for in vivo administration, and allow for delivery of salinomycin at a greater dose, e.g., at least 1.0 mg/kg. In some embodiments, an aqueous composition comprising 0.01-1.5 mg/mL salinomycin, 0.5-5% (v/v) ethanol, 1-10% (v/v) propylene glycol, and 1-20% (v/v) Solutol® HS 15 is well tolerated in test subjects, as described below in the Examples. This composition can be administered in test subjects at 0.5 to 1.5 mg/kg intravenously without obvious side effects from the intravenous dose. Exemplary side effects include dizziness, discolored urine, elevated pulse, hyperactivity, rapid breathing, lowered pulse, sluggishness, or difficulty breathing.

### Methods of Administration and Dosage

The aqueous compositions of this invention may be administered orally, parenterally, by inhalation spray, topically, rectally, nasally, buccally, vaginally or via an implanted reservoir, preferably by oral administration or administration by injection. In some cases, the pH of the aqueous composition may be adjusted with pharmaceutically acceptable acids, bases or buffers to enhance the stability or efficacy of the aqueous composition.

In some embodiments, the compositions described herein may be administered by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion with or without further dilution.

In some embodiments the aqueous composition is administered to a subject parenterally. In some embodiments, the aqueous compositions are dosed intravenously at a dose of 0.001 to 10 mg/kg, e.g., 0.005 to 5 mg/kg, e.g., 0.01 to 1 mg/kg, e.g., 0.1 to 1 mg/kg, e.g., 0.1, or 0.2, or 0.3, or 0.4, or 0.5, or 0.6, or 0.7, or 0.8, or 0.9, or 1.0 mg/kg. In some embodiments, the subject is administered the aqueous composition orally. In some embodiments, the aqueous compositions are dosed orally at a dose of 0.01 to 100 mg/kg, e.g., 0.05 to 50 mg/kg, e.g., 0.1 to 10 mg/kg, e.g., 1 to 10 mg/kg, e.g., 2, or 2, or 3, or 4, or 5, or 6, or 7, or 8, or 9, or 10 mg/kg. The term parenteral as used herein includes subcutaneous, intracutaneous, intravenous, intramuscular, intraarticular, intraarterial, intrasynovial, intrasternal, intrathecal, intralesional and intracranial injection or infusion techniques. In some embodiments, the aqueous composition is configured for intravenous administration.

In some embodiments the aqueous composition is be orally administered in any orally acceptable dosage form including, but not limited to, oral dosage form, syrups, emulsions and aqueous suspensions. Additional thickening agents, for example gums, e.g., xanthum gum, starches, e.g., corn starch, or glutens may be added to achieve a desired consistency of the aqueous composition when used as an oral dosage. If desired, certain sweetening and/or flavoring and/or coloring agents may be added.

In some embodiments, the method further comprises administering an additional cancer treatment e.g., radiation therapy, chemotherapy, or hormone therapy in combination with the aqueous composition. In some embodiments, the additional cancer treatment is administered simultaneously with the formulation. In some embodiments, the additional cancer treatment is administered sequentially with the formulation. In some embodiments the additional cancer treatment is chemotherapy. In some embodiments, the chemotherapy is a taxane, e.g., docitaxel, paclitaxel, or cabazitaxel. In some embodiments, the chemotherapy is a platinum compound, e.g., cisplatin. In some embodiments, the chemotherapy is a PARP inhibitor, e.g., inaparib. In some embodiments, the chemotherapy is an anthracycline, e.g., doxorubicin.

Subject doses of the aqueous compositions described herein typically range from about 0.1 μg to 10,000 mg, more typically from about 1 g to 8000 mg, e.g., from about 10 μg to 100 mg once or more per day, week, month, or other time interval. Stated in terms of subject body weight, typical dosages in certain embodiments of the invention range from about 0.1 μg to 20 mg/kg/day, e.g., from about 1 to 10 mg/kg/day, e.g., from about 1 to 5 mg/kg/day. In some embodiments, the aqueous compositions are dosed intravenously at a dose of 0.001 to 10 mg/kg, e.g., 0.005 to 5 mg/kg, e.g., 0.01 to 1 mg/kg, e.g., 0.1 to 1 mg/kg, e.g., 0.1, or 0.2, or 0.3, or 0.4, or 0.5, or 0.6, or 0.7, or 0.8, or 0.9, or 1.0 mg/kg. In some embodiments, the aqueous compositions are dosed orally at a dose of 0.01 to 100 mg/kg, e.g., 0.05 to 50 mg/kg, e.g., 0.1 to 10 mg/kg, e.g., 1 to 10 mg/kg, e.g., 2, or 2, or 3, or 4, or 5, or 6, or 7, or 8, or 9, or 10 mg/kg. The absolute amount will depend upon a variety of factors including the concurrent treatment, the number of doses and the individual subject parameters including age, physical condition, size and weight. These are factors well known to those of ordinary skill in the art and can be addressed with no more than routine experimentation. It is often the case that a maximum dose be used, that is, the highest safe dose according to sound medical judgment. The dose used may be the maximal tolerated dose or a sub-therapeutic dose or any dose there between. Multiple doses of the molecules of the invention are also contemplated. When the molecules of the invention are administered in combination a sub-therapeutic dosage of either of the molecules, or a sub-therapeutic dosage of both, may be used in the treatment of a subject having, or at risk of developing, cancer. When the two classes of drugs are used together, the cancer medicament may be administered in a sub-therapeutic dose to produce a desirable therapeutic result. A sub-therapeutic dose is a dosage which is less than that dosage which would produce a therapeutic result in the subject if administered in the absence of the other agent. Thus, the sub-therapeutic dose of a cancer medicament is one which would not produce the desired therapeutic result in the subject in the absence of the administration of the molecules of the invention. Therapeutic doses of cancer medicaments are well known in the field of medicine for the treatment of cancer. These dosages have been extensively described in references such as Remington's Pharmaceutical Sciences, 18th ed., 1990; as well as many other medical references relied upon by the medical profession as guidance for the treatment of cancer.

Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

In some embodiments, the aqueous compositions described herein are incorporated into a dosage form. In some embodiments, the dosage form is a parenteral dosage form, e.g., for administration to a subject intravenously. In some embodiments, the dosage form is composition in a sterile, sealed container (e.g., a bottle, a vial). In some embodiments, the dosage form may be an oral dosage form, e.g., for administration to a subject orally. In some embodiments, an oral dosage form additionally comprises flavors, or fragrances, or both, to modify the taste or odor of the oral dosage form.

### Methods of Treatment

### Proliferative Disorders

Compositions (e.g., aqueous compositions) described herein may be used for treating, e.g., ameliorating, alleviating, curing, maintaining a cure (i.e., delaying relapse) of a proliferative disorder, such as cancer. A “proliferative disorder” is a disease or disorder characterized by cells that have the capacity for autonomous growth or replication, e.g., an abnormal state or condition characterized by proliferative cell growth. Exemplary proliferative disorders include solid tumors and cancers of the blood, for example, carcinoma, sarcoma, metastatic disorders (e.g., tumors arising from prostate, colon, lung, breast and liver origin), hematopoietic proliferative disorders, e.g., leukemias, metastatic tumors. Prevalent cancers include: breast, prostate, colon, lung, liver, and pancreatic cancers. Treatment with the aqueous composition may be in an amount effective to ameliorate at least one symptom of the proliferative disorder, e.g., reduced cell proliferation, reduced tumor mass, etc.

The disclosed methods are useful in the treatment of cancer, including for example, solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer, Astrocytoma, Childhood Cerebellar; Astrocytoma, Childhood Cerebral; Bile Duct Cancer, Extrahepatic; Bladder Cancer, Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer, Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primaiy; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer, Colorectal Cancer, Childhood; Cutaneous T-Cell Lymphoma; Endometrial Cancer, Ependymoma, Childhood; Epithelial Cancer, Ovarian; Esophageal Cancer, Esophageal Cancer, Childhood; Ewing's Family of Tumors; Extracranial Germ Cell Tumor, Childhood; Extragonadal Germ Cell Tumor, Extrahepatic Bile Duct Cancer; Eye Cancer, Intraocular Melanoma; Eye Cancer, Retinoblastoma; Gallbladder Cancer, Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer, Hypothalamic and Visual Pathway Glioma, Childhood; Intraocular Melanoma; Islet Cell Carcinoma (Endocrine Pancreas); Kaposi's Sarcoma; Kidney Cancer, Laryngeal Cancer, Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS—Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer, Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer, Nasopharyngeal Cancer, Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer, Oropharyngeal Cancer, Osteosarcoma/Malignant Fibrous Histiocytoma of Bone; Ovarian Cancer, Childhood; Ovarian Epithelial Cancer, Ovarian Germ Cell Tumor, Ovarian Low Malignant Potential Tumor; Pancreatic Cancer; Pancreatic Cancer, Childhood; Pancreatic Cancer, Islet Cell; Paranasal Sinus and Nasal Cavity Cancer, Parathyroid Cancer, Penile Cancer, Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer, Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer, Renal Cell (Kidney) Cancer, Renal Cell Cancer, Childhood; Renal Pelvis and Ureter, Transitional Cell Cancer, Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer, Small Intestine Cancer, Soft Tissue Sarcoma, Adult; Soft Tissue Sarcoma, Childhood; Squamous Neck Cancer with Occult Primary, Metastatic; Stomach (Gastric) Cancer, Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer, Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer, Visual Pathway and Hypothalamic Glioma, Childhood; Vulvar Cancer, Waldenstrom's Macro globulinemia; and Wilms' Tumor. Metastases of the aforementioned cancers can also be treated or minimized in accordance with the methods described herein.

In some embodiments, the cancer is, or is characterized as comprising or enriched for, cancer stem cells (CSCs), tumor-initiating cells, mesenchymal cells, or mesenchymal-like cells associated with cancer, or mesenchymal cancerous cells. For example, the aqueous compositions can be administered to a subject to kill, inhibit the growth of, limit the proliferation of, or cause other beneficial changes to a subject (e.g., a human) having cancer. In some embodiments, the cancer is associated with CSCs, or tumor-initiating cells, mesenchymal cells, or mesenchymal-like cells associated with cancer, or mesenchymal cancerous cells, or the cancer is characterized as being enriched with CSCs or mesenchymal cells (e.g, a CSC-enriched tumor, a tumor with mesenchymal cells, or a tumor with cells that have undergone an epithelial-to-mesenchymal transition). In an embodiment, treatment with a composition, e.g., an aqueous composition, described herein, may reduce the spread of cancer, e.g., a metastatic cancer. In an embodiment, treatment with a composition, e.g., an aqueous composition, described herein, may reduce the likelihood of relapse of a cancer, e.g., reducing the likelihood of self-initiation of a new tumor. In embodiments where treatment has started after a diagnosis with a disorder, the aqueous compositions and methods described herein can reduce, ameliorate or altogether eliminate the disorder, and/or its associated symptoms, to keep it from becoming worse, to slow the rate of progression, or to minimize the rate of recurrence of the disorder once it has been initially eliminated (i.e., to avoid a relapse). A suitable dose and therapeutic regimen may vary depending upon the specific composition used, the mode of delivery of the compound, and whether it is used alone or in combination. As used herein, a therapeutically effective amount is an amount of an aqueous composition that inhibits cancer (e.g., a CSC-enriched tumor, a tumor with mesenchymal cells, or a tumor with cells that have undergone an epithelial-to-mesenchymal transition) formation, progression, and/or spread (e.g., metastasis). A therapeutically effective amount can refer to any one or more of the compounds or compositions described herein, or discovered using the methods described herein, that have CSC-enriched tumor inhibitory properties (e.g, inhibit the growth and/or survival of CSCs, or cancerous mesenchymal cells). The effective amount of an aqueous composition described herein can vary depending on such factors as the cancer being treated, the size of the subject, or the severity and/or progression of the disease or condition. In some embodiments a useful composition increases the average length of survival, increases the average length of progression-free survival, and/or reduces the rate of recurrence, of subjects treated with the aqueous composition in a statistically significant manner. In some embodiments, an aqueous composition described herein is used to inhibit the growth or differentiation of a cancer stem cell or cancerous mesenchymal cell, e.g., by contacting the cancer stem cell or cancerous mesenchymal cell with an aqueous salinomycin composition. The contacting may take place in vitro or in vivo. In some embodiments, the cancer stem cell or cancerous mesenchymal cell has or is characterized by activity in a transcription factor selected from Snail1, Snail2, Goosecoid, FoxC1, FoxC2, TWIST, E2A, SIP-1/Zeb-2, dEF1/Zeb1, LEF, Myc, HMGA2, TAZ, Klf8, HIF-1, HOXB7, SIM2s, and Fos. In some embodiments, the cancer stem cell or cancerous mesenchymal cell has or is characterized by activity in a pathway selected from TGF-β, Wnt, BMP, Notch, HGF-Met, EGF, IGF, PDGF, FGF, P38-mapk, Ras, PB Kinase-Akt, Src, and NF-kB. In some embodiments, the aqueous compositions described herein can be administered to cells in culture, e.g. in vitro or ex vivo, or to a subject, e.g., in vivo, to treat, modulate, and/or diagnose a variety of disorders, including those described herein below.

### Cancer Combination Therapies

In certain embodiments, the aqueous compositions described herein may be taken alone or in combination with other therapeutics. In one embodiment, a mixture of two or more compositions may be administered to a subject in need thereof. In yet another embodiment, one or more compositions may be administered with one or more therapeutic agents for the treatment or avoidance of various diseases, including, for example, cancer, diabetes, neurodegenerative diseases, cardiovascular disease, blood clotting, inflammation, flushing, obesity, aging, stress, etc. In various embodiments, combination therapies comprising a salinomycin composition may refer to (1) pharmaceutical compositions that comprise one or more compositions in combination with one or more therapeutic agents (e.g., one or more therapeutic agents described herein); and (2) co-administration of one or more salinomycin compositions with one or more therapeutic agents wherein the salinomycin composition and therapeutic agent have not been formulated in the same compositions (but may be present within the same kit or package, such as a blister pack or other multi-chamber package; connected, separately sealed containers (e.g., foil pouches) that can be separated by the user; or a kit where the salinomycin composition(s) and other therapeutic agent(s) are in separate vessels). When using separate compositions, the salinomycin compositions may be administered at the same time as, intermittently, staggered, prior to, subsequent to, or combinations thereof, with respect to the administration of another therapeutic agent.

In some embodiments, an aqueous composition described herein is administered together with an additional cancer treatment. Exemplary cancer treatments include, for example: chemotherapy, antibiotics, targeted therapies such as antibody therapies, immunotherapy, and hormonal therapy. For example, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antibiotics, enzymes, epipodophyllotoxins, platinum coordination complexes, vinca alkaloids, substituted ureas, methyl hydrazine derivatives, adrenocortical suppressants, hormone antagonists, enzyme inhibitors, endostatin, taxols, camptothecins, doxorubicins and their analogs, and combinations thereof.

Examples of each of these treatments are provided below.

Chemotherapy

In some embodiments, an aqueous composition described herein is administered with a chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, and streptozocin), platinum agents (i.e., alkylating-like agents) (e.g., carboplatin, cisplatin, oxaliplatin, BBR3464, and satraplatin), busulfan, dacarbazine, procarbazine, temozolomide, thioTEPA, treosulfan, and uramustine; antimetabolites such as folic acids (e.g., aminopterin, methotrexate, pemetrexed, and raltitrexed); purines such as cladribine, clofarabine, fludarabine, mercaptopurine, pentostatin, and thioguanine; pyrimidines such as capecitabine, cytarabine, fluorouracil, floxuridine, and gemcitabine; spindle poisons/mitotic inhibitors such as taxanes (e.g., docetaxel, paclitaxel, cabazitaxel) and vincas (e.g., vinblastine, vincristine, vindesine, and vinorelbine); cytotoxic/antitumor antibiotics such anthracyclines (e.g., daunorubicin, doxorubicin, epirubicin, idarubicin, mitoxantrone, pixantrone, and valrubicin), compounds naturally produced by various species of streptomyces (e.g., actinomycin, bleomycin, mitomycin, plicamycin) and hydroxyurea; topoisomerase inhibitors such as camptotheca (e.g., camptothecin, topotecan and irinotecan) and podophyllums (e.g., etoposide, teniposide); monoclonal antibodies for cancer immunotherapy such as anti-receptor tyrosine kinases (e.g., cetuximab, panitumumab, trastuzumab), anti-CD20 (e.g., rituximab and tositumomab), and others for example alemtuzumab, bevacizumab, and gemtuzumab; photosensitizers such as aminolevulinic acid, methyl aminolevulinate, porfimer sodium, and verteporfin; tyrosine kinase inhibitors such as cediranib, dasatinib, erlotinib, gefitinib, imatinib, lapatinib, nilotinib, sorafenib, sunitinib, and vandetanib; serine/threonine kinase inhibitors, (e.g., inhibitors of AbI, c-Kit, insulin receptor family member(s), EGF receptor family member(s), Akt, mTOR (e.g., rapamycin or analogs thereof, direct inhibitors of mTORC1 and/or mTORC2), Raf kinase family, phosphatidyl inositol (PI) kinases such as PI3 kinase, PI kinase-like kinase family members, cyclin dependent kinase family members, aurora kinase family), growth factor receptor antagonists, and others such as retinoids (e.g., alitretinoin and tretinoin), altretamine, amsacrine, anagrelide, arsenic trioxide, asparaginase (e.g., pegaspargase), bexarotene, bortezomib, denileukin diftitox, estramustine, ixabepilone, masoprocol, mitotane, and testolactone, Hsp90 inhibitors, proteasome inhibitors, HDAC inhibitors, angiogenesis inhibitors, e.g., anti-vascular endothelial growth factor agents such as, bevacizumab or VEGF-Trap, matrix metalloproteinase inhibitors, pro-apoptotic agents (e.g., apoptosis inducers), anti-inflammatory agents, etc.

Because some drugs work better together than alone, two or more drugs are often given at the same time or sequentially. Often, two or more chemotherapy agents are used as combination chemotherapy. In some embodiments, the chemotherapy agents (including combination chemotherapy) can be used in combination with an aqueous composition described herein. In some embodiments, an aqueous composition described herein may be administered with another chemotherapeutic and another compound identified as being effective in the treatment or modulation of proliferation of cancer stem cells.

Targeted Therapy

In some embodiments, an aqueous composition described herein is administered with a targeted therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. One example is tyrosine kinase inhibitors, e.g., a kinase inhibitor listed above, monoclonal antibody therapies, e.g., therapeutics comprising an antibody which specifically binds to a protein on the surface of the cancer cells, e.g., a monoclonal antibody therapy listed herein. Another example is PARP inhibitors, i.e., pharmacological inhibitors of the enzyme poly ADP ribose polymerase (PARP). Suitable PARP inhibitors may be iniparib, olaparib, rucaparib, veliparib, or CEP 9722. In some embodiments, the targeted therapy can be used in combination with an aqueous composition described herein. Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell.

Immunotherapy

In some embodiments, an aqueous composition described herein is administered with an immunotherapy. Cancer immunotherapy refers to a diverse set of therapeutic strategies designed to induce the subject's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma subjects.

Allogeneic hematopoietic stem cell transplantation can be considered a form of immunotherapy, since the donor's immune cells will often attack the tumor in a graft-versus-tumor effect. In some embodiments, the immunotherapy agents can be used in combination with an aqueous composition described herein.

Hormonal Therapy

In some embodiments, an aqueous composition described herein is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. Examples of hormonal therapies include tamoxifen (Nolvadex®, Istubal®, Valodex®), abarelix (Plenaxis®), flutamide (Eulexin®), bicalutamide (Casodex®), nilutamide (Nilandron®), an degarelix (Firmagon®). In some embodiments, the hormonal therapy agents can be used in combination with an aqueous composition described herein.

Radiation Therapy

The formulations described herein can be used in combination with directed energy or particle, or radioisotope treatments, e.g., radiation therapies, e.g., radiation oncology, for the treatment of proliferative disease, e.g., cancer, e.g., cancer associated with cancer stem cells. The formulations may be administered to a subject simultaneously or sequentially along with the directed energy or particle, or radioisotope treatments. For example, the formulations may be administered before, during, or after the directed energy or particle, or radioisotope treatment, or a combination thereof. The directed energy or particle therapy may comprise total body irradiation, local body irradiation, or point irradiation. The directed energy or particle may originate from an accelerator, synchrotron, nuclear reaction, vacuum tube, laser, or from a radioisotope. The therapy may comprise external beam radiation therapy, teletherapy, brachytherapy, sealed source radiation therapy, systemic radioisotope therapy, or unsealed source radiotherapy. The therapy may comprise ingestion of, or placement in proximity to, a radioisotope, e.g., radioactive iodine, cobalt, cesium, potassium, bromine, fluorine, carbon. External beam radiation may comprise exposure to directed alpha particles, electrons (e.g., beta particles), protons, neutrons, positrons, or photons (e.g., radiowave, millimeter wave, microwave, infrared, visible, ultraviolet, X-ray, or gamma-ray photons). The radiation may be directed at any portion of the subject in need of treatment. The radiation may also be administered to cultured cells or cell samples, i.e., in vitro radiation therapy. In one embodiment, the cultured cells are cultured cancer stem cells.

Surgery

The aqueous compositions described herein can be used in combination with surgery, e.g., surgical exploration, intervention, biopsy, for the treatment of proliferative disease, e.g., cancer, e.g., cancer associated with cancer stem cells. The aqueous compositions may be administered to a subject simultaneously or sequentially along with the surgery. For example, the aqueous compositions may be administered before (pre-operative), during, or after (post-operative) the surgery, or a combination thereof. The surgery may be a biopsy during which one or more cells are collected for further analysis. The biopsy may be accomplished, for example, with a scalpel, a needle, a catheter, an endoscope, a spatula, or scissors. The biopsy may be an excisional biopsy, an incisional biopsy, a core biopsy, or a needle biopsy, e.g., a needle aspiration biopsy. The surgery may involve the removal of localized tissues suspected to be or identified as being cancerous. For example, the procedure may involve the removal of a cancerous lesion, lump, polyp, or mole. The procedure may involve the removal of larger amounts of tissue, such as breast, bone, skin, fat, or muscle. The procedure may involve removal of part of, or the entirety of, an organ or node, for example, lung, throat, tongue, bladder, cervix, ovary, testicle, lymph node, liver, pancreas, brain, eye, kidney, gallbladder, stomach, colon, rectum, or intestine. In one embodiment, the cancer is breast cancer, e.g., triple negative breast cancer, and the surgery is a mastectomy or lumpectomy.

### Microbial Disorders

An aqueous composition described herein can be used to treat a microbial growth or disorder. A “microbial disorder” is a disease or disorder characterized by growth of foreign cells on or within a subject, for example by a bacteria, virus, or fungus. The aqueous composition may target the cell wall or cell membrane of the microbes, or interfere with essential pathways thereby limiting the growth of the microbe. Exemplary microbial disorders include infection by coccidia, Staphylococcus aureus, Enterococcus faecalis and Enterococcus faecium, Streptococcus pneumoniae, E. coli, Salmonella, Klebsiella pneumoniae, Pseudomonas species and Enterobacter species.

### Microbial Combination Therapies

In some embodiments, a composition described herein is administered together with another antibiotic, e.g, a cephalosporin, a penicillin, a quinolone, a sulfonamide, or a tetracycline. Suitable antibiotics include abacavir, acyclovir, albendazole, amikacin, amoxicillin, ampicillin, azithromycin, aztreonam, benzilpenicillin, cefepime, ceftriaxone, cephalexin, chloramphenicol, chloroquine, cilastatin, clindamycin, co-trimoxazole, didanosine, dioxidine, doxycycline, famciclovir, fluconazole, fosfomycin, furazolidone, fusidic acid, ganciclovir, gentamicin, isoniazid, josamycin, kanamycin, ketoconazole, lamivudine, lincomycin, linezolid, mebendazole, meropenem, metronidazole, moxifloxacin, mupirocin, nystatin, nitrofuranton, nitroxoline, norfloxacin, ofloxacin, ornidazole, oseltamivir, polymixin B, polymyxin M, proguanil, ribavirin, rifampicin, rimantadine, roxithromycin, spectinomycin, sulfodimidin, teicoplanin, terbinafine, tetracycline, timidazole, valaciclovir, valganciclovir, vancomycin, zanamivir, or zidovudine. One class of antibiotics, known as ionophores, includes lonomycin, ionomycin, laidlomycin, nigericin, grisorixin, dianemycin, lenoremycin, salinomycin, narasin, alborixin, septamycin, maduramicin, semduramicin, lasalocid, mutalomycin, isolasalocid A, lysocellin, tetronasin, and echeromycin.

### Subject Selection and Monitoring

In some embodiments, described herein, e.g., a subject suffering from or suspected of suffering from a disorder described herein or a sample taken from the subject, may be tested for the presence of a biomarker, e.g., one or more biomarkers associated with a cancer, e.g., a cancer stem cell, or a biomarker indicative of the presence of mesenchymal cells, prior to being administered compounds described herein. In some embodiments, the aqueous compositions are administered to a subject that has been identified with a predictive biomarker indicating the prevalence of CSCs, or tumor-initiating cells, or mesenchymal cells, or mesenchymal-like cells associated with cancer, or mesenchymal cancerous cells, or wherein the cancer is characterized as being enriched with CSCs or mesenchymal cells.

In order to identify or evaluate the biomarker, e.g., a cancer stem cell biomarker, or a biomarker indicative of the presence of mesenchymal cells, it may be necessary to obtain a clinical sample from the subject (e.g., a sample of the cancer). Typically, a clinical sample is a tumor biopsy or cells isolated there from. However, the invention is not so limited and any suitable clinical sample may be used, provided that the sample has a detectable cancer stem cell biomarker in a subject having a cancer stem cell. Exemplary clinical samples include saliva, hair folicles, gingival secretions, cerebrospinal fluid, gastrointestinal fluid, mucus, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions.

In one embodiment, the clinical sample is screened for a genetic marker indicative of a disorder suitable for treatment with the compounds described herein, or for the presence of one or more genes correlated with a risk for developing a disorder suitable for treatment with the compounds described herein. For example, gene expression analysis (e.g., nucleic acid microarray, cDNA array, quantitative RT-PCR, RNAse protection assay) can be employed to identify specific genes or to locate markers indicative of genes related to the disorder. In some embodiments, one or more of the following genes, may be identified: ANAPC2, CCND1 (cyclin D1), CCNE1 (cyclin E1), CDC7, CDC34, CDK4, CDK6, CDKNIB (p27), CDKNIC (p57), CDKN3, CUL1, CUL2, CUL3, CUL4A, CUL5, E2F1, SKP2; S Phase and DNA Replication: ABL1 (C-ABL), MCM2, MCM3, MCM4 (CDC21), MCM5 (CDC46), MCM6 (Mis5), MCM7 (CDC47), PCNA, RP A3, SUMO1, UBE1; G2 Phase and G2/M Transition: ANAPC2, ANAPC4, ANAPC5, ARHI, BCCIP, BIRC5, CCNA1 (cyclin A1), CCNB1 (cyclin B1), CCNG1 (cyclin G1), CCNH, CCNT1, CCNT2, CDC25A, CDC25C, CDC37, CDK5R1, CDK5R2, CDK5RAP1, CDK5RAP3, CDK2, CDK7, CDKN3, CKS1B, CKS2, DDXl 1, DNM2, GTF2H1, GTSE1, HERCS, KPNA2, MNAT1, PKMYT1, RGC32, SERTAD1; M Phase: CCNB2 (cyclin B2), CCNF, CDC2 (CDK1), CDC 6, CDC20 (p55cdc), CDC25A, CDC25C, MRE1 IA, RAD50, RAD51; Cell Cycle Checkpoint and Cell Cycle Arrest: ATM, ATR, BRCA1, BRC A2, CCNA2 (cyclin A2), CCNE2 (cyclin E2), CCNG2 (cyclin G2), CDC2 (CDK1), CDC25A, CDC34, CDC45L, CDC6, CDK2, CDKN1A (p21), CDKN1B (p27), CDKNIC (p57), CDKN2A (p16), CDKN2B (p15), CDKN2C (p18), CDKN2D (p19), CDKN3, CHEK1 (CHK1), CHEK2 (CHK2/RAD53), CUL1, CUL2, CUL3, CUL4A, CUL5, GADD45A, HUS1, KNTC1, MAD2L1, MAD2L2, NBS1 (NIBRIN), RAD1, RAD17, RAD9A, RB1, RBBP8, TP53 (p53); Regulation of the Cell Cycle: ABL1 (C-ABL), ANAPC2, ANAPC4, ANAPC5, ARHI, ATM, ATR, BCCIP, BCL2, BRCA2, CCNA1 (cyclin A1), CCN A2 (cyclin A2), CCNB1 (cyclin B1), CCNB2 (cyclin B2), CCNC (cyclin C), CCND1 (cyclin D1), CCND2 (cyclin D2), CCND3 (cyclin D3), CCNE1 (cyclin E1), CCNE2 (cyclin E2), CCNF (cyclin F), CCNH (cyclin H), CCNT1, CCNT2, CDC 16, CDC2 (CDK1), CDC20 (p55cdc), CDC25A, CDC25C, CDC37, CDC45L, CDC6, CDK2, CDK4, CDK5R1, CDK5R2, CDK6, CDK7, CDK8, CDKN1A (p21), CDKN1B (p27), CKS1B, CUL5, DDXl 1, E2F1, E2F2, E2F3, E2F4, E2F5, E2F6, GADD45A, KNTC1, MKI67 (Ki67), PCNA, PKMYT1, RAD9A, RB1, SKP2, TFDP1 (DPI), TFDP2 (DP2); Negative Regulation of the Cell Cycle: ATM, BAX, BRCA1, CDC7, CDKN2B (p15), CDKN2D (p19), RBL1 (p1O7 RB), RBL2 (p130 RB2), TP53 (p53). Exemplary Cell Survival/Apoptotic Genes include those of the TNF Ligand Family: LTA (TNF-α), TNF (TNF-a), TNFSF5 (CD40 Ligand), TNFSF6 (FasL), TNFSF7 (CD27 Ligand), TNFSF8 (CD30 Ligand), TNFSF9 (4-IBB Ligand), TNFSF1O (TRAIL), TNFSF14 (HVEM-L), TNFSF18; the TNF Receptor Family: LTBR, TNFRSF1A (TNFR1), TNFRSF1B (TNFR2), TNFRSF5 (CD40), TNFRSF6 (Fas), TNFRSF6B, TNFRSF7 (CD27), TNFRSF9 (4-1BB), TNFRSF1OA (DR4), TNFRSF1OB (DR5), TNFRSF1OC (DcR1), TNFRSF1OD (DcR2), TNFRSF1 IB, TNFRSF 12A, TNFRSF 14 (HVEM), TNFRSF 19, TNFRSF21, TNFRSF25; the Bcl-2 Family: BAD, BAG1, BAG3, BAG4, BAK1, BAX, BCL2, BCL2A1 (bfl-1), BCL2L1 (bcl-x), BCL2L2 (bcl-w), BCL2L10, BCL2L11 (bim-like protein), BCL2L12, BCL2L13, BCLAF1, BID, BIK, BNIP1, BNIP2, BNIP3 (nip3), BNIP3L, BOK (Mtd), HRK, MCL1; the Caspase Family: CASP1, CASP2, CASP3, CASP4, CASP5, CASP6, CASP7, CASP8, CASP9, CASP1O, CASP 14; the IAP Family: BIRC1 (NIAP), BIRC2 (IAP2), BIRC3 (IAP1), BIRC4 (XIAP), BIRC5 (Survivin), BIRC6 (Bruce), BIRC7, BIRC8; the TRAF Family: TRAF1, TRAF2, TRAF3 (CRAF1), TRAF4, TRAF5; the CARD Family: APAF1, BCL1O (HuE1O), BIRC2, BIRC3, CARD4 (NOD1), CARD6, CARD8, CARD9, CARD1O, CARD11, CARD12, CARD14, CARD 15, CASP1, CASP2, CASP4, CASP5, CASP9, CRADD, NOL3 (Nop30), PYCARD, RIPK2 (CARDIAC); the Death Domain Family: CRADD, DAPK1, DAPK2, FADD, RIPK1, TNFRSF1OA, TNFRSF1OB, TNFRSF1 IB, TNFRSF1A, TNFRSF21, TNFRSF25, TNFRSF6, TRADD; the CIDE Domain Family: CIDEA, CIDEB, DFFA, DFFB; the p53 and DNA Damage Response: ABL1, AKT1, APAF1, BAD, BAX, BCL2, BCL2L1, BID, CASP3, CASP6, CASP7, CASP9, GADD45A, TP53 (p53), TP53BP2, TP73, TP73L; and AKT1, BAG1, BAG3, BAG4, BCL2, BCL2A1, BCL2L1, BCL2L10, BCL2L2, BFAR, BIRC1, BIRC2, BIRC3, BIRC4, BIRC5, BIRC6, BIRC7, BIRC8, BNIP1, BNIP2, BNIP3, BRAF, CASP2, CFLAR, GDNF, IGF1R, MCL1, TNF (TNF-a), TNFRSF6, TNFRSF6B, TNFRSF7, TNFSF 18, TNFSF5.

In one embodiment, a stem cell biomarker, or a biomarker indicative of the presence of mesenchymal cells, is selected from E-cadherin, TWIST expression, and a CD44/CD24 cell surface marker profile. The stem cell biomarker, or the biomarker indicative of the presence of mesenchymal cells, may be identified in a sample of a cancer obtained from the subject. In one embodiment, the E-cadherin and/or TWIST expression in the cancer is determining by measuring a level of E-cadherin and/or TWIST protein and/or RNA expression in the cancer, and optionally comparing the level to a reference standard. In one embodiment, the reference standard is the level of E-cadherin and/or TWIST protein and/or RNA expression in a cancer stem cell. In one embodiment, the reference standard is the level of E-cadherin and/or TWIST protein and/or RNA expression in a cancer cell that is not a cancer stem cell.

In one embodiment, the stem cell biomarker, or a biomarker indicative of the presence of mesenchymal cells, is selected from CD20, CD24, CD34, CD38, CD44, CD45, CD105, CD133, CD166, EpCAM, ESA, SCA1, Pecam, and Stro1.

In some cases it may be desirable to evaluate a cancer stem cell biomarker, or a biomarker indicative of the presence of mesenchymal cells, in a subject having, or suspect of having, cancer, and to select a treatment for the subject based on the results of the biomarker evaluation. For example, if the cancer stem cell biomarker, or the biomarker indicative of the presence of mesenchymal cells, is detected, the subject may be treated with an effective amount of an aqueous composition disclosed herein. In some embodiments, if the cancer stem cell biomarker, or the biomarker indicative of the presence of mesenchymal cells, is detected, the subject may be treated with an effective amount of a pharmaceutical composition comprising abamectin, etoposide or nigericin, or a derivative of any of the foregoing, optionally in combination with paclitaxel or a derivative thereof (e.g., water-soluble or targeted derivatives or structurally related compounds, e.g., analogs such as docetaxel (see, e.g., WO/2003/045932 and US2008033189). The cancer stem cell biomarker, or the biomarker indicative of the presence of mesenchymal cells, of the foregoing methods may be evaluated using methods disclosed herein or any suitable methods known in the art. Exemplary cancer stem cell biomarkers, or biomarkers indicative of the presence of mesenchymal cells, include E-cadherin expression, TWIST expression, and a CD44⁺CD24 marker profile. Other biomarkers may indicate activity in a pathway selected from TGF-β, Wnt, BMP, Notch, HGF-Met, EGF, IGF, PDGF, FGF, P38-mapk, Ras, PB Kinase-Akt, Src, and NF-kB. Other exemplary cancer stem cell biomarkers, or biomarkers indicative of the presence of mesenchymal cells, are disclosed herein and will be apparent to one of ordinary skill in the art.

In one embodiment, the clinical sample may be screened for protein levels, for example the level of protein encoded by a cell cycle/growth and/or survival gene, e.g., a gene listed above. Protein levels can be assessed by an appropriate method known to one of ordinary skill in the art, such as western analysis. Other methods known to one of ordinary skill in the art could be employed to analyze proteins levels, for example immunohistochemistry, immunocytochemistry, ELISA, radioimmunoassays, and proteomics methods, such as mass spectroscopy or antibody arrays.

After having been identified with a biomarker, a subject receiving an aqueous composition described herein can be monitored, for example, for improvement in the condition and/or adverse effects, or for the expression of biomarkers indicative of the disorder. Improvement of a subject's condition can be evaluated, for example, by monitoring the growth, absence of growth, or regression of the cancer (e.g., a tumor). In some embodiments, the subject is evaluated using a radiological assay or evaluation of hemolytic parameters. In other embodiments the subject may be evaluated using gene or protein assays described herein. The subject may also be evaluated using conventional screening methods, such as physical exam, mammography, biopsy, colonoscopy, etc.

### Kits

The invention additionally includes kits comprising compositions described herein. In some embodiments, the kit additionally comprises a diluent for the purpose of diluting the aqueous composition as it is received in the kit. In some embodiments, the diluent is water. In some embodiments, the diluent is a pharmaceutically-acceptable vehicle, e.g., a vehicle disclosed herein. In some embodiments, the diluent comprises water. In some embodiments the diluent comprises at least 1% miscible organic solvent (v/v), e.g., at least 5%, at least 10%, at least 15%, at least 20%, at least 25%, at least 30%, at least 35%, at least 40% miscible organic solvent (v/v). In some embodiments, the diluent comprises at least 0.1% solubilizer and/or emulsifying agent (v/v), e.g., at least 0.5%, at least 1%, at least 5%, at least 10%, at least 15%, at least 20%, at least 25%, at least 30% solubilizer and/or emulsifying agent (v/v). In one embodiment, the diluent comprises about 5% of miscible organic solvent and about 5% of solubilizer and/or emulsifying agent, the balance being water. In some embodiments, the kit comprises instructions for diluting the aqueous composition with the diluent included in the kit.

In some embodiments, the kit comprises an additionally therapeutic agent, e.g., a chemotherapeutic, e.g., a chemotherapeutic agent described herein. In some embodiments, the kit additionally comprises instructions for administering the aqueous composition along with the additional therapeutic agent.

The aqueous compositions described herein may be administered to a subject as an aqueous composition or dosage form. In some cases the aqueous compositions or dosage forms may be part of a kit, along with instructions for administering the aqueous composition. The kit may additionally comprise a diluent (e.g., water, saline, or a vehicle described herein) and instructions for administering the diluents along with the aqueous composition as intended. The aqueous compositions may be administered along with additional therapeutic agents, if present, in amounts effective for achieving a modulation of disease or disease symptoms, including those described herein. The additional therapeutic agents may be administered simultaneous with the aqueous compositions described herein, or they may be administered sequentially with the aqueous compositions described herein.

### EXAMPLES

### Example A

### Aqueous Salinomycin Sodium Compositions

A number of salinomycin compositions were prepared and evaluated for suitability as injectable compositions. For the aqueous compositions described below, crude salinomycin sodium was purchased from Zhejiang Shenghua Baike Pharmaceutical (China) and further purified to >95% purity prior to being incorporated into the aqueous compositions. Generally, the purified salinomycin sodium was dissolved in a miscible organic solvent (ethanol, propylene glycol, or DMSO; Fisher Scientific), an emulsifying agent was optionally added (Solutol® HS 15 [BASF], Tween® 80 [Sigma-Aldrich], Cremophor® EL [BASF]) with mixing, and then deionized water was added to achieve the desired concentration. As shown in Table 1, below, only some of the aqueous compositions resulted in clear solutions, which were deemed suitable for use as injectable compositions.

TABLE 1
Aqueous salinomycin sodium compositions and the resulting properties.

| | Concentration of | | |
|---|---|---|---|
| | salinomycin | | Resulting |
| No. | sodium (mg/mL) | Carrier composition (v/v) | solution |
| 1 | 50.0 | 40% Ethanol/60% DIW | Clear |
| 2 | 50.0 | 40% Propylene Glycol/60% DIW | Milky |
| 3 | 50.0 | 50% Solutol HS 15/50% DIW | Insoluble |
| 4 | 50.0 | 5% Ethanol/10% Solutol HS 15/85% DIW | Milky |
| 5 | 50.0 | 5% Ethanol/15% Solutol HS 15/80% DIW | Clear |
| 6 | 50.0 | 10% Ethanol/10% Solutol HS 15/80% DIW | Clear |
| 7 | 50.0 | 15% Ethanol/50% Solutol HS 15/35% DIW | Clear |
| 8 | 50.0 | 15% PG/10% Solutol HS 15/75% DIW | Milky |
| 9 | 50.0 | 20% PG/20% Solutol HS 15/60% DIW | Clear |
| 10 | 50.0 | 5% Ethanol/10% PG/10% Solutol HS 15/75% DIW | Milky |
| 11 | 50.0 | 5% Ethanol/10% PG/20% Solutol HS 15/65% DIW | Clear |
| 12 | 50.0 | 10% Ethanol/10% PG/20% Solutol HS 15/60% DIW | Clear |
| 13 | 50.0 | 10% Ethanol/20% Tween 80/70% DIW | Milky |
| 14 | 50.0 | 10% Ethanol/30% Tween 80/60% DIW | Milky |
| 15 | 50.0 | 15% Ethanol/10% Tween 80/75% DIW | Milky |
| 16 | 50.0 | 20% Ethanol/30% Tween 80/50% DIW | Clear |
| 17 | 50.0 | 20% PG/30% Tween 80/50% DIW | Milky |
| 18 | 50.0 | 10% Ethanol/10% PG/30% Tween 80/50% DIW | Insoluble |
| 19 | 50.0 | 5% Ethanol/5% Cremophor EL/90% DIW | Milky |
| 20 | 50.0 | 5% Ethanol/10% Cremophor EL/85% DIW | Milky |
| 21 | 50.0 | 10% Ethanol/10% Cremophor EL/80% DIW | Clear |
| 22 | 50.0 | 5% DMSO/5% Cremophor EL/90% DIW | Milky |
| 23 | 50.0 | 5% DMSO/10% Cremophor EL/85% DIW | Milky |
| 24 | 50.0 | 10% DMSO/10% Cremophor EL/80% DIW | Milky |
| 25 | 50.0 | 2.5% Ethanol/2.5% DMSO/5% Cremophor EL/90% DIW | Milky |
| 26 | 50.0 | 2.5% Ethanol/2.5% DMSO/10% Cremophor EL/85% DIW | Milky |
| 27 | 50.0 | 5% Ethanol/5% DMSO/10% Cremophor EL/80% DIW | Clear |
| 28 | 50.0 | 5% DMSO/5% Solutol HS 15/90% DIW | Milky |
| 29 | 50.0 | 5% DMSO/10% Solutol HS 15/85% DIW | Milky |
| 30 | 50.0 | 10% DMSO/10% Solutol HS 15/80% DIW | Clear |
| 31 | 50.0 | 2.5% Ethanol/2.5% DMSO/5% Solutol HS 15/90% DIW | Milky |
| 32 | 50.0 | 2.5% Ethanol/2.5% DMSO/10% Solutol HS 15/85% DIW | Milky |
| 33 | 50.0 | 5% Ethanol/5% DMSO/10% Solutol HS 15/80% DIW | Clear |
| 34 | 10.0 | 1% Ethanol/3% Solutol HS 15/96% DIW | Insoluble |
| 35 | 10.0 | 2% Ethanol/5% Solutol HS 15/93% DIW | Clear |
| 36 | 10.0 | 3% Ethanol/4% Solutol HS 15/93% DIW | Clear |
| 37 | 10.0 | 3% PG/10% Solutol HS 15/87% DIW | Clear |
| 38 | 10.0 | 5% PG/10% Solutol HS 15/85% DIW | Clear |
| 39 | 10.0 | 10% PG/10% Solutol HS 15/80% DIW | Clear |
| 40 | 10.0 | 0.5% Ethanol/2% PG/2.5% Solutol HS 15/95% DIW | Insoluble |
| 41 | 10.0 | 1% Ethanol/4% PG/5% Solutol HS 15/90% DIW | Clear |
| 42 | 10.0 | 2% Ethanol/8% PG/5% Solutol HS 15/85% DIW | Clear |
| 43 | 10.0 | 5% Ethanol/10% Tween 80/85% DIW | Milky |
| 44 | 10.0 | 10% Ethanol/10% Tween 80/80% DIW | Milky |
| 45 | 10.0 | 12% Ethanol/10% Tween 80/78% DIW | Milky |
| 46 | 10.0 | 15% Ethanol/10% Tween 80/75% DIW | Milky |
| 47 | 10.0 | 10% PG/10% Tween 80/80% DIW | Milky |
| 48 | 10.0 | 15% PG/10% Tween 80/75% DIW | Milky |
| 49 | 10.0 | 20% PG/10% Tween 80/70% DIW | Milky |
| 50 | 10.0 | 25% PG/10% Tween 80/65% DIW | Milky |
| 51 | 10.0 | 30% PG/10% Tween 80/60% DIW | Milky |
| 52 | 10.0 | 2% Ethanol/4% PG/10% Tween 80/84% DIW | Milky |
| 53 | 10.0 | 4% Ethanol/8% PG/10% Tween 80/78% DIW | Milky |
| 54 | 10.0 | 5% Ethanol/10% PG/10% Tween 80/75% DIW | Milky |
| 55 | 10.0 | 8% Ethanol/10% PG/10% Tween 80/72% DIW | Milky |
| 56 | 10.0 | 10% Ethanol/12% PG/10% Tween 80/68% DIW | Milky |
| 57 | 10.0 | 10% Ethanol/20% PG/10% Tween 80/60% DIW | Clear |

### Example B

### Tolerability of Various Vehicle Compositions

Eight aqueous vehicle compositions were prepared by mixing ethanol and/or propylene glycol (Fisher Scientific) with an emulsifying agent (Solutol® HS 15 [BASF] or Tween® 80 [Sigma-Aldrich]) and then adding deionized water to achieve the desired concentration. The aqueous vehicle compositions are shown in Table O below. Each aqueous composition was administered to two ICR mice (Sino-British SIPPR/BK Lab Animal Ltd, Shanghai, China). All mice had a body weight within ±20% of the mean body weight for the study. The individual animal number was marked on the tail, and study number comprised a unique identification for each animal. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily. Each mouse was observed immediately after dosage with the vehicle and then at days 2, 3, and 4 post dosage.

TABLE O
Aqueous vehicle compositions

| Vehicle | | |
|---|---|---|
| Composition | Composition (v/v) | Observations |
| 5 | 5% Ethanol/15% Solutol HS 15/80% DIW | No abnormalities |
| 6 | 10% Ethanol/10% Solutol HS 15/80% DIW | No abnormalities |
| 9 | 20% Propylene Glycol/20% Solutol HS 15/60% | Convulsions and decreased |
| | DIW | activity; one mouse died |
| 11 | 5% Ethanol/10% Propylene Glycol/20% | No abnormalities |
| | Solutol HS 15/65% DIW | |
| 16 | 20% Ethanol/30% Tween 80/50% DIW | Rapid breathing, grasping |
| | | decreased, skin cold to touch |
| 37 | 3% Propylene Glycol/10% Solutol HS 15/87% | No abnormalities |
| | DIW | |
| 41 | 1% Ethanol/4% Propylene Glycol/5% Solutol | No abnormalities |
| | HS 15/90% DIW | |
| 57 | 10% Ethanol/20% Propylene Glycol/10% | Briefly laying prostrate; no |
| | Tween 80/60% DIW | other abnormalities detected |

Based upon the results depicted in Table O, vehicle compositions 1 (10% Ethanol/10% Solutol HS 15/80% DIW), 2 (5% Ethanol/15% Solutol HS 15/80% DIW), 4 (3% Propylene Glycol/10% Solutol HS 15/87% DIW), 6 (1% Ethanol/4% Propylene Glycol/5% Solutol HS 15/90% DIW), and 7 (5% Ethanol/10% Propylene Glycol/20% Solutol HS 15/65% DIW) were tolerated acceptably in mouse test subjects.

### Example C

### 50 mg/mL Salinomycin Sodium in Ethanol, Solutol® HS 15, and Water

50.0 mg of salinomycin sodium was dissolved in 2.0 mL of ethanol (Fisher Scientific). To this solution was added 2.0 mL of Solutol® HS 15 (BASF) with stirring. Finally, dionized water was added to make 20 total mL of solution. The resulting solution was clear and colorless.

### Example D

50 mg/mL Salinomycin Sodium in Ethanol, Solutol® HS 15, and Water

50.0 mg of salinomycin sodium was dissolved in 1.0 mL of ethanol (Fisher Scientific). To this solution was added 3.0 mL of Solutol® HS 15 (BASF) with stirring. Finally, dionized water was added to make 20 total mL of solution. The resulting solution was clear and colorless.

### Example E

### 50 mg/mL Salinomycin Sodium in Ethanol, Propylene Glycol, Solutol® HS 15, and Water

50.0 mg of salinomycin sodium was dissolved in 2.0 mL of ethanol (Fisher Scientific). To this solution was added 4.0 mL of Solutol® HS 15 (BASF) with stirring. In a separate container, 3.0 mL of propylene glycol (Fisher Scientific) was added to 20.0 mL of dionized water to make a PG-water solution. The PG-water solution was then added to the mixture of salinomycin sodium, ethanol, and Solutol® HS 15 to make 20 total mL of solution. The resulting solution was clear and colorless.

### Example F

### 10 mg/mL Salinomycin Sodium in Propylene Glycol, Solutol® HS 15, and Water

10.0 mg of salinomycin sodium was dissolved in 0.6 mL of propylene glycol (Fisher Scientific). To this solution was added 2.0 mL of Solutol® HS 15 (BASF) with stirring. Finally, dionized water was added to make 20 total mL of solution. The resulting solution was clear and colorless.

### Example G

### 10 mg/mL Salinomycin Sodium in Ethanol, Propylene Glycol, Solutol® HS 15, and Water

10.0 mg of salinomycin sodium was dissolved in 0.2 mL of ethanol (Fisher Scientific). To this solution was added 1.0 mL of Solutol® HS 15 (BASF) with stirring. In a separate container, 0.8 mL of propylene glycol (Fisher Scientific) was added to 20.0 mL of dionized water to make a PG-water solution. The PG-water solution was then added to the mixture of salinomycin sodium, ethanol, and Solutol® HS 15 to make 20 total mL of solution. The resulting solution was clear and colorless.

### Example H

### Tolerability of 0.3, 0.6, and 1.0 Mg/Kg Salinomycin Sodium in 1% Ethanol/4% Propylene Glycol/5% Solutol® HS 15 Vehicle Administered Intravenously

Injectable salinomycin compositions were prepared as follows: A) 0.2 mg/mL composition: 1.01 mg of salinomycin sodium was added to 50 μL ethanol and vortexed for 1 minute to yield a clear solution. 200 μL of propylene glycol was then added and vortexed for 1 minute, yielding a clear solution. To this solution was next added 250 μL Solutol® HS 15, with vortexing for 1 minute and sonicating for 1 minute, to yield a clear solution. Finally, 4.5 mL water was added, giving a final solution that was clear and colorless (pH˜7). B) 0.12 mg/mL composition: 2.4 mL of the 0.2 mg/mL composition (previous) was added to 1.6 mL mixed blank vehicle (1% ethanol/4% propylene glycol/5% Solutol® HS 15/90% water) to yield a final concentration of 0.12 mg/mL; the resulting solution was clear and colorless (pH=7). C) 0.06 mg/mL composition: 1.5 mL solution of the 0.12 mg/mL composition (previous) was added to 1.5 mL mixed blank vehicle to yield a final concentration of 0.06 mg/mL; the resulting solution is clear and colorless (pH=7).

Eight male ICR mice (Sino-British SIPPR/BK Lab Animal Ltd, Shanghai, China) were assigned to control and treatment groups. Each mouse had a body weight within ±20% of the mean body weight for the study. The individual animal number marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 5 consecutive days. The dose levels for the treated groups and the control group are in the below table. Individual doses were based on the most recent body weight.

TABLE Q
Dosage schedule for evaluating tolerability of 0.3, 0.6, and 1.0 mg/kg
intravenously

| | | | Dose | Dose | Dose | Dose |
|---|---|---|---|---|---|---|
| Group | Number of | | Level | Concentration | Volume | Route |
| Number | Animals | Test Article | (mg/kg) | (mg/mL) | (mL/kg) | & Frequency |
| 1 | 2 | vehicle | 0 | 0 | 5 | IV, 5 days |
| 2 | 2 | Salinomycin | 0.3 | 0.06 | 5 | IV, 5 days |
| 3 | 2 | Salinomycin | 0.6 | 0.12 | 5 | IV, 5 days |
| 4 | 2 | Salinomycin | 1 | 0.2 | 5 | IV, 5 days |

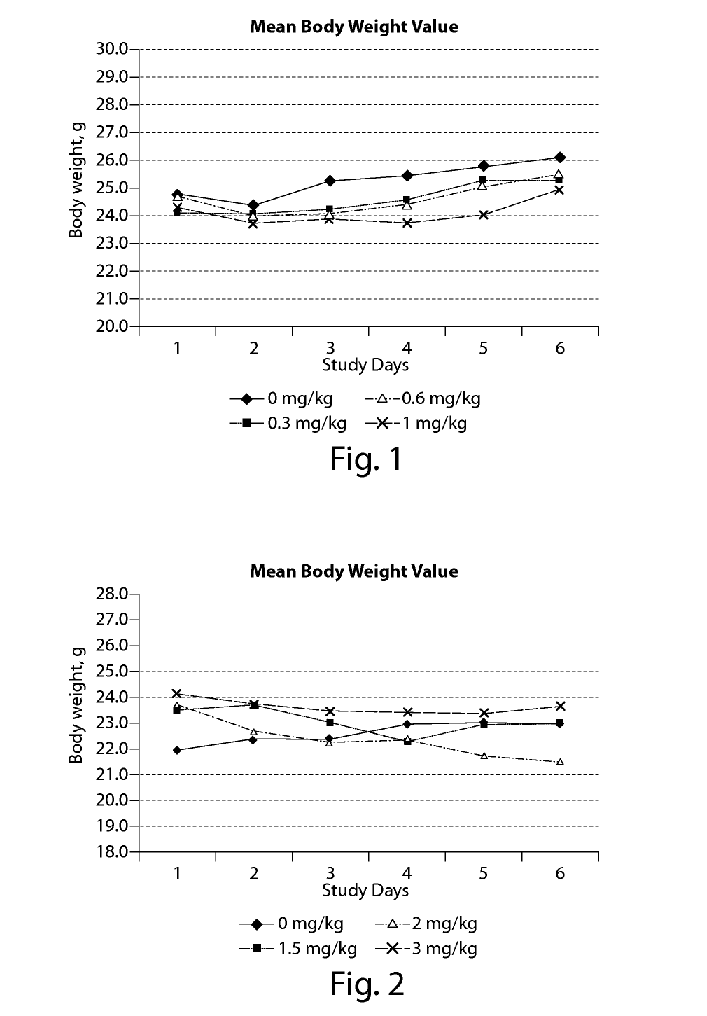

Body weights for all animals were measured and recorded at receipt, prior to randomization, and once daily during the study. Body weight change was calculated for mean value of animals relative to Day 1 mean body weight value. As shown in FIG. 1, all groups added weight during the study. Furthermore, there were no abnormalities were detected in any of the study animals, suggesting that 1 mg/kg can be safely dosed.

### Example I

### Tolerability of 1.5, 2.0, 3.0 Mg/Kg Salinomycin Sodium in 1% Ethanol/4% Propylene Glycol/5% Solutol® HS 15 Vehicle Administered Intravenously

Injectable salinomycin compositions were prepared as follows: A) 0.6 mg/mL composition: 3.03 mg of salinomycin sodium was added to 50 μL ethanol and vortexed for 1 minute to yield a clear solution. 200 μL of propylene glycol was then added and vortexed for 1 minute, yielding a clear solution. To this solution was next added 250 μL Solutol® HS 15, with vortexing for 1 minute and sonicating for 1 minute, to yield a clear solution. Finally, 4.5 mL water was added, giving a final solution that was clear and colorless (pH˜7). B) 0.4 mg/mL composition: 2.6 mL of the 0.6 mg/mL composition (previous) was added to 1.4 mL mixed blank vehicle (1% ethanol/4% propylene glycol/5% Solutol® HS 15/90% water) to yield a final concentration of 0.4 mg/mL; the resulting solution was clear and colorless (pH=7). C) 0.3 mg/mL composition: 1.5 mL solution of the 0.6 mg/mL composition (previous) was added to 1.5 mL mixed blank vehicle to yield a final concentration of 0.3 mg/mL; the resulting solution is clear and colorless (pH=7).

Eight male ICR mice (Sino-British SIPPR/BK Lab Animal Ltd, Shanghai, China) were assigned to control and treatment groups. Each mouse had a body weight within ±20% of the mean body weight for the study. The individual animal number marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 5 consecutive days. The dose levels for the treated groups and the control group are in the below table. Individual doses were based on the most recent body weight.

TABLE R
Dosage schedule for evaluating tolerability of 1.5, 2.0, 3.0 mg/kg intravenously

| | | | Dose | Dose | Dose | Dose |
|---|---|---|---|---|---|---|
| Group | Number of | | Level | Concentration | Volume | Route |
| Number | Animals | Test Article | (mg/kg) | (mg/mL) | (mL/kg) | & Frequency |
| 1 | 2 | vehicle | 0 | 0 | 5 | IV, 5 days |
| 2 | 2 | Salinomycin | 1.5 | 0.3 | 5 | IV, 5 days |
| 3 | 2 | Salinomycin | 2 | 0.4 | 5 | IV, 5 days |
| 4 | 2 | Salinomycin | 3 | 0.6 | 5 | IV, 5 days |

Body weights for all animals were measured and recorded at receipt, prior to randomization, and once daily during the study. Body weight change was calculated for mean value of animals relative to Day 1 mean body weight value. As shown in FIG. 2, the weight of all groups were stable with the exception of the 2 mg/kg group which lost weight during the study. The animals dosed at 1.5 mg/kg were observed to have decreased activity levels (e.g., more time spent laying prostrate), however, they returned to normal function at the conclusion of the study. The animals dosed at 2.0 mg/kg were observed to have decreased activity and rapid breathing post dosage. One of the animals dosed at 2.0 mg/kg was found dead on day 4 of the study. The animals dosed at 3.0 mg/kg were observed to have decreased activity, rapid breathing and loss of reflex activity after dosing. There were no abnormalities detected in the control group, e.g., the vehicle dosed group.

### Example J

### Tolerability of 5.0, 10.0 Mg/Kg Salinomycin Sodium in 1% Ethanol/4% Propylene Glycol/5% Solutol® HS 15 Vehicle Administered Orally

Fompositions for oral administration were prepared as follows: A) 1.0 mg/mL composition: 5.05 mg of salinomycin sodium was added to 50 μL ethanol and vortexed for 1 minute to yield a clear solution. 200 μL of propylene glycol was then added and vortexed for 1 minute, yielding a clear solution. To this solution was next added 250 μL Solutol® HS 15, with vortexing for 1 minute and sonicating for 1 minute, to yield a clear solution. Finally, 4.5 mL water was added, giving a final solution that was clear and colorless (pH˜7). B) 0.5 mg/mL composition: 1.5 mL solution of the 1.0 mg/mL composition (previous) was added to 1.5 mL mixed blank vehicle to yield a final concentration of 0.5 mg/mL; the resulting solution is clear and colorless (pH=7).

Six male ICR mice (Sino-British SIPPR/BK Lab Animal Ltd, Shanghai, China) were assigned to control and treatment groups. Each mouse had a body weight within ±20% of the mean body weight for the study. The individual animal number marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via oral gavage for 5 consecutive days. The dose levels for the treated groups and the control group are in the below table. Individual doses were based on the most recent body weight.

TABLE S
Dosage schedule for evaluating tolerability of 5.0, 10.0 mg/kg orally

| | | | Dose | Dose | Dose | Dose |
|---|---|---|---|---|---|---|
| Group | Number of | | Level | Concentration | Volume | Route |
| Number | Animals | Test Article | (mg/kg) | (mg/mL) | (mL/kg) | & Frequency |
| 1 | 2 | vehicle | 0 | 0 | 10 | PO, 5 days |
| 2 | 2 | Salinomycin | 5 | 0.5 | 10 | PO, 5 days |
| 3 | 2 | Salinomycin | 10 | 1 | 10 | PO, 5 days |

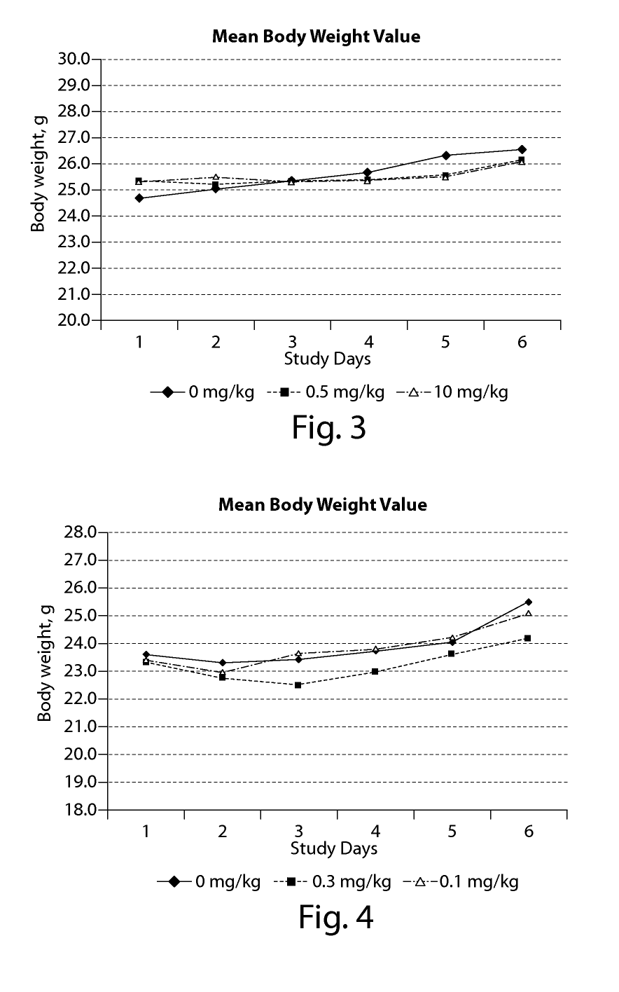

Body weights for all animals were measured and recorded at receipt, prior to randomization, and once daily during the study. Body weight change was calculated for mean value of animals relative to Day 1 mean body weight value. As shown in FIG. 3, all groups added weight during the study. Furthermore, there were no abnormalities were detected in any of the study animals, suggesting that the maximum oral tolerated dosage is at least 10 mg/kg.

### Example K

### Tolerability of 0.02, 0.06 Mg/Kg Salinomycin Sodium in 10% Ethanol/10% Solutol® HS 15 Vehicle Administered Intravenously

Injectable salinomycin compositions were prepared as follows: 0.6 mg/mL stock solution: 1.2 mg of salinomycin sodium was added to 200 μL ethanol and vortexed for 2 minutes and sonicated for 30 seconds to yield a clear solution. To this solution was next added 200 μL Solutol® HS 15, with vortexing for 2 minutes. Finally, 1.6 mL water was added to produce a clear stock solution of 0.6 mg/mL salinomycin sodium. A) 0.06 mg/mL composition: 200 μL of the stock solution was mixed with 1.8 mL of blank vehicle (10% ethanol/10% Solutol® HS 15/80% water) to yield a final concentration of 0.06 mg/mL; the resulting solution was clear and colorless (pH=7). B) 0.02 mg/mL composition: 500 μL of solution A) was mixed with 1.0 mL of blank vehicle (10% ethanol/10% Solutol® HS 15/80% water) to yield a final concentration of 0.02 mg/mL; the resulting solution was clear and colorless (pH=7).

Six male ICR mice (Sino-British SIPPR/BK Lab Animal Ltd, Shanghai, China) were assigned to control and treatment groups. Each mouse had a body weight within ±20% of the mean body weight for the study. The individual animal number marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 5 consecutive days. The dose levels for the treated groups and the control group are in the below table. Individual doses were based on the most recent body weight.

TABLE T
Dosage schedule for evaluating tolerability of 0.02, 0.06 mg/kg intravenously.

| | | | Dose | Dose | Dose | Dose |
|---|---|---|---|---|---|---|
| Group | Number of | | Level | Concentration | Volume | Route & |
| Number | Animals | Test Article | (mg/kg) | (mg/mL) | (mL/kg) | Frequency |
| 1 | 2 | vehicle | 0 | 0 | 5 | IV, 5 days |
| 2 | 2 | Salinomycin | 0.1 | 0.02 | 5 | IV, 5 days |
| 3 | 2 | Salinomycin | 0.3 | 0.06 | 5 | IV, 5 days |

Body weights for all animals were measured and recorded at receipt, prior to randomization, and once daily during the study. Body weight change was calculated for mean value of animals relative to Day 1 mean body weight value. As shown in FIG. 4, all groups added weight during the study. One animal dosed with the vehicle was observed with vocalization at day 2. The animals dosed at 0.1 mg/kg were observed to have decreased activity (e.g., more time spent laying prostrate), rapid breathing, and partially-closed eyelids post dosage. The animals dosed at 0.3 mg/kg were observed to have decreased activity levels, partially-closed eyelids, and skin cold to the touch post dosage.

### Example L

### Tolerability of 0.02, 0.06 Mg/Kg Salinomycin Sodium in 5% Ethanol/15% Solutol® HS 15 Vehicle Administered Intravenously

Injectable salinomycin compositions were prepared as follows: 0.6 mg/mL stock solution: 1.2 mg of salinomycin sodium was added to 100 μL ethanol and vortexed for 2 minutes and sonicated for 2 minutes to yield a clear solution. To this solution was next added 300 μL Solutol® HS 15, with vortexing for 1 minute. Finally, 1.6 mL water was added to produce a clear stock solution of 0.6 mg/mL salinomycin sodium. A) 0.06 mg/mL composition: 200 μL of the stock solution was mixed with 1.8 mL of blank vehicle (5% ethanol/15% Solutol® HS 15/80% water) to yield a final concentration of 0.06 mg/mL; the resulting solution was clear and colorless (pH=7). B) 0.02 mg/mL composition: 500 μL of solution A) was mixed with 1.0 mL of blank vehicle (10% ethanol/10% Solutol® HS 15/80% water) to yield a final concentration of 0.02 mg/mL; the resulting solution was clear and colorless (pH=7).

Six male ICR mice (Sino-British SIPPR/BK Lab Animal Ltd, Shanghai, China) were assigned to control and treatment groups. Each mouse had a body weight within ±20% of the mean body weight for the study. The individual animal number marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 5 consecutive days. The dose levels for the treated groups and the control group are in the below table. Individual doses were based on the most recent body weight.

TABLE U
Dosage schedule for evaluating tolerability of
0.02, 0.06 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 5 | IV, 5 days |
| 2 | 2 | Salinomycin | 0.1 | 0.02 | 5 | IV, 5 days |
| 3 | 2 | Salinomycin | 0.3 | 0.06 | 5 | IV, 5 days |

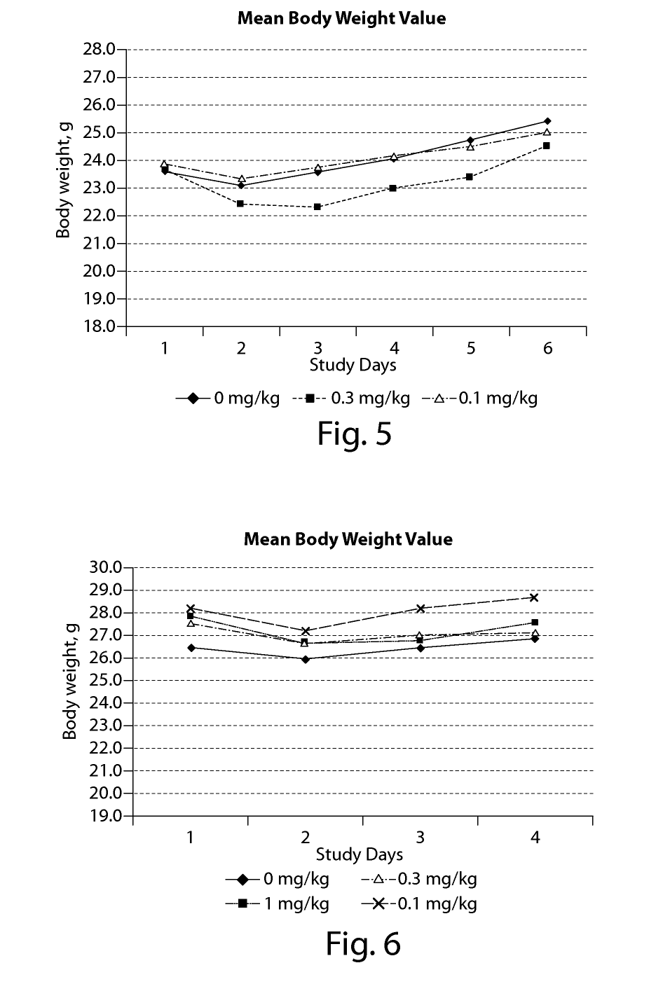

Body weights for all animals were measured and recorded at receipt, prior to randomization, and once daily during the study. Body weight change was calculated for mean value of animals relative to Day 1 mean body weight value. As shown in FIG. 5, all groups added weight during the study. One animal dosed at 0.1 mg/kg was observed to have decreased activity immediately after dosing on day 1. One animal dosed at 0.3 mg/kg was observed to have hypersensitivity to touch after dosing on day 1. There were no abnormalities detected in the control group, e.g., the vehicle dosed group.

### Example M

### Tolerability of 0.1, 0.3, 1.0, 3.0 Mg/Kg Salinomycin Sodium in 10% Ethanol/10% Solutol® HS 15 Vehicle Administered Intravenously

Using the techniques described above in Example B, an aqueous vehicle comprising 10% ethanol (v/v) and 10% Solutol® HS 15 (v/v) was prepared. An appropriate amount of salinomycin sodium was dissolved into the vehicle to make solutions having concentrations of 0.02, 0.06, 0.2, and 0.3 mg/mL. Ten male ICR mice (Sino-British SIPPR/BK Lab Animal Ltd, Shanghai, China) were assigned to control and treatment groups. Each mouse had a body weight within ±20% of the mean body weight for the study. The individual animal number was marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 4 consecutive days. The dose levels for the treated groups and the control group are in the below table. Individual doses were based on the most recent body weight.

TABLE V
Dosage schedule for evaluating tolerability of 0.1, 0.3,
1.0, 3.0 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 10 | IV, 4 days |
| 2 | 2 | Salinomycin | 0.1 | 0.02 | 5 | IV, 4 days |
| 3 | 2 | Salinomycin | 0.3 | 0.06 | 5 | IV, 4 days |
| 4 | 2 | Salinomycin | 1 | 0.2 | 5 | IV, 4 days |
| 5 | 2 | Salinomycin | 3 | 0.3 | 10 | IV, 4 days |

Body weights for all animals were measured and recorded at receipt, prior to randomization, and once daily during the study. Body weight change was calculated for mean value of animals relative to Day 1 mean body weight value. As shown in FIG. 6, the weight of each group surviving administration was relatively stable during the study. The animals dosed at 0.1 mg/kg showed no abnormalities throughout the study. The animals dosed at 0.3 mg/kg were observed to have skin cold to the touch after the first administration, however this condition dissipated after 3 hours and there were no abnormalities on days 2, 3, or 4. The animals dosed at 1.0 mg/kg were observed to have decreased activity, decreased grasping, and skin cold to the touch after the first administration, but no abnormalities on days 2, 3, or 4. The animals dosed at 3.0 mg/kg died shortly after administration. There were no abnormalities detected in the control group, e.g., the vehicle dosed group.

### Example N

### Tolerability of 0.1, 03, 1.0, 3.0 Mg/Kg Salinomycin Sodium in 5% Ethanol/15% Solutol® HS 15 Vehicle Administered Intravenously

Using the techniques described above in Example A, an aqueous vehicle comprising 5% ethanol (v/v) and 15% Solutol® HS 15 (v/v) was prepared. An appropriate amount of salinomycin sodium was dissolved into the vehicle to make solutions having concentrations of 0.02, 0.06, 0.2, and 0.3 mg/mL. Ten male ICR mice (Sino-British SIPPR/BK Lab Animal Ltd, Shanghai, China) were assigned to control and treatment groups. Each mouse had a body weight within ±20% of the mean body weight for the study. The individual animal number was marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 4 consecutive days. The dose levels for the treated groups and the control group are in the below table. Individual doses were based on the most recent body weight.

TABLE W
Dosage schedule for evaluating tolerability of 0.1, 0.3,
1.0, 3.0 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 10 | IV, 4 days |
| 2 | 2 | Salinomycin | 0.1 | 0.02 | 5 | IV, 4 days |
| 3 | 2 | Salinomycin | 0.3 | 0.06 | 5 | IV, 4 days |
| 4 | 2 | Salinomycin | 1 | 0.2 | 5 | IV, 4 days |
| 5 | 2 | Salinomycin | 3 | 0.3 | 10 | IV, 4 days |

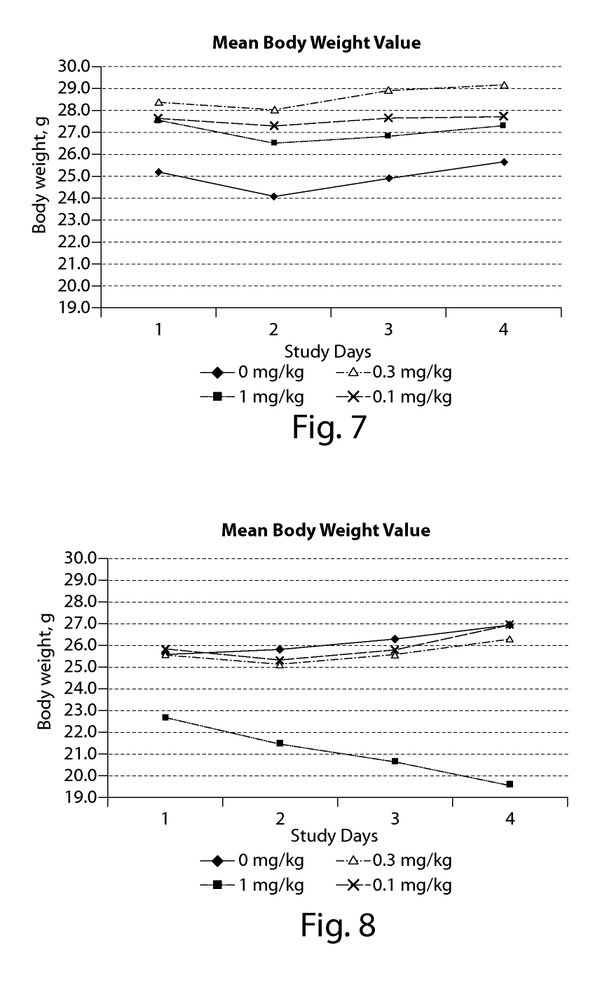

Body weights for all animals were measured and recorded at receipt, prior to randomization, and once daily during the study. Body weight change was calculated for mean value of animals relative to Day 1 mean body weight value. As shown in FIG. 7, the weight of each group surviving administration was relatively stable during the study. The animals dosed at 0.1 mg/kg showed no abnormalities throughout the study. The animals dosed at 0.3 mg/kg showed no abnormalities throughout the study. The animals dosed at 1.0 mg/kg were observed to have decreased activity, decreased grasping, and laying prostrate after the first administration, but no abnormalities on days 2, 3, or 4. The animals dosed at 3.0 mg/kg died shortly after administration. There were no abnormalities detected in the control group, e.g., the vehicle dosed group.

### Example O

### Tolerability of 0.1, 03, 1.0 Mg/Kg Salinomycin Sodium in 3% Propylene Glycol/10% Solutol® HS 15 Vehicle Administered Intravenously

Using the techniques described above in Example A, an aqueous vehicle comprising 3% propylene glycol (v/v) and 10% Solutol® HS 15 (v/v) was prepared. An appropriate amount of salinomycin sodium was dissolved into the vehicle to make solutions having concentrations of 0.02, 0.06, and 0.2 mg/mL. Eight male ICR mice (Sino-British SIPPR/BK Lab Animal Ltd, Shanghai, China) were assigned to control and treatment groups. Each mouse had a body weight within ±20% of the mean body weight for the study. The individual animal number was marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 4 consecutive days. The dose levels for the treated groups and the control group are in the below table. Individual doses were based on the most recent body weight.

TABLE X
Dosage schedule for evaluating tolerability of 0.1, 0.3,
1.0 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 5 | IV, 4 days |
| 2 | 2 | Salinomycin | 0.1 | 0.02 | 5 | IV, 4 days |
| 3 | 2 | Salinomycin | 0.3 | 0.06 | 5 | IV, 4 days |
| 4 | 2 | Salinomycin | 1 | 0.2 | 5 | IV, 4 days |

Body weights for all animals were measured and recorded at receipt, prior to randomization, and once daily during the study. Body weight change was calculated for mean value of animals relative to Day 1 mean body weight value. As shown in FIG. 8, the weight of each groups administered the control and the 0.1 and 0.3 mg/kg doses remained relatively stable during the study. The weight of the animals receiving the 1 mg/kg dosage decreased steadily through the trial. The animals dosed at 0.1 mg/kg showed no abnormalities throughout the study. The animals dosed at 0.3 mg/kg were observed to have a slight decrease in activity after initial administration, but no abnormalities on days 2, 3, or 4. The animals dosed at 1.0 mg/kg were observed to have decreased activity and leaning, but no abnormalities on days 2, 3, or 4. There were no abnormalities detected in the control group, e.g., the vehicle dosed group.

### Example P

### Tolerability of 0.1, 0.3, 1.0 Mg/Kg Salinomycin Sodium in 1% Ethanol/4% Propylene Glycol/5% Solutol® HS 15 Vehicle Administered Intravenously

Using the techniques described above in Example A, an aqueous vehicle comprising 1% ethanol (v/v), 4% propylene glycol (v/v) and 5% Solutol® HS 15 (v/v) was prepared. An appropriate amount of salinomycin sodium was dissolved into the vehicle to make solutions having concentrations of 0.02, 0.06, and 0.2 mg/mL. Eight male ICR mice (Sino-British SIPPR/BK Lab Animal Ltd, Shanghai, China) were assigned to control and treatment groups. Each mouse had a body weight within ±20% of the mean body weight for the study. The individual animal number was marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 4 consecutive days. The dose levels for the treated groups and the control group are in the below table. Individual doses were based on the most recent body weight.

TABLE Y
Dosage schedule for evaluating tolerability of
0.1, 0.3, 1.0 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 5 | IV, 4 days |
| 2 | 2 | Salinomycin | 0.1 | 0.02 | 5 | IV, 4 days |
| 3 | 2 | Salinomycin | 0.3 | 0.06 | 5 | IV, 4 days |
| 4 | 2 | Salinomycin | 1 | 0.2 | 5 | IV, 4 days |

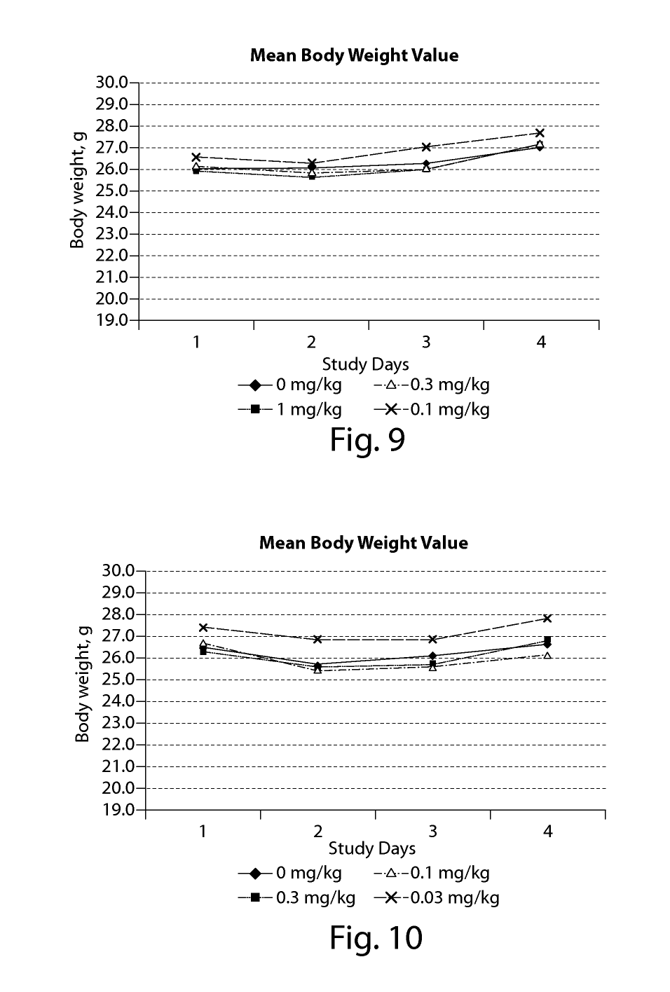

Body weights for all animals were measured and recorded at receipt, prior to randomization, and once daily during the study. Body weight change was calculated for mean value of animals relative to Day 1 mean body weight value. As shown in FIG. 9, the weight of each group increased during the study. The animals dosed at 0.1 mg/kg showed no abnormalities throughout the study. The animals dosed at 0.3 mg/kg showed no abnormalities throughout the study. The animals dosed at 1 mg/kg were observed to have a decrease in activity after initial administration, but no abnormalities on days 2, 3, or 4. There were no abnormalities detected in the control group, e.g., the vehicle dosed group.

### Example Q

### Tolerability of 0.03, 0.1, 03, 1.0 Mg/Kg Salinomycin Sodium in 5% Ethanol/10% Propylene Glycol/20% Solutol HS® 15 Vehicle Administered Intravenously

Using the techniques described above in Example A, an aqueous vehicle comprising 5% ethanol (v/v), 10% propylene glycol (v/v), and 10% Solutol® HS 15 (v/v) was prepared. An appropriate amount of salinomycin sodium was dissolved into the vehicle to make solutions having concentrations of 0.006, 0.02, 0.06, and 0.2 mg/mL. Ten male ICR mice (Sino-British SIPPR/BK Lab Animal Ltd, Shanghai, China) were assigned to control and treatment groups. Each mouse had a body weight within ±20% of the mean body weight for the study. The individual animal number was marked on the tail, and study number comprised a unique identification for each animal. Animal identification was verified during the course of the study. The animals were housed two per cage in stainless steel suspended cage in an environmentally controlled room. SLACOM (Rodent Diet #20110615029, Shanghai SLAC Laboratory Animal Co. Ltd.) was available ad libitum. Temperature and humidity were monitored and recorded twice daily.

Each composition and the vehicle (control) were administered once daily to each of two mice via intravenous bolus injection for 4 consecutive days. The dose levels for the treated groups and the control group are in the below table. Individual doses were based on the most recent body weight.

TABLE Z
Dosage schedule for evaluating tolerability of 0.03, 0.1,
0.3, 1.0, 3.0 mg/kg intravenously.

| | | | Dose | Dose | | |
|---|---|---|---|---|---|---|
| | Number | | Level | Con- | Dose | Dose |
| Group | of | | (mg/ | centration | Volume | Route & |
| Number | Animals | Test Article | kg) | (mg/mL) | (mL/kg) | Frequency |
| 1 | 2 | vehicle | 0 | 0 | 5 | IV, 4 days |
| 2 | 2 | Salinomycin | 0.03 | 0.006 | 5 | IV, 4 days |
| 3 | 2 | Salinomycin | 0.1 | 0.02 | 5 | IV, 4 days |
| 4 | 2 | Salinomycin | 0.3 | 0.06 | 5 | IV, 4 days |
| 5 | 2 | Salinomycin | 1 | 0.2 | 5 | IV, 4 days |

Body weights for all animals were measured and recorded at receipt, prior to randomization, and once daily during the study. Body weight change was calculated for mean value of animals relative to Day 1 mean body weight value. As shown in FIG. 10, the weight of each group surviving administration was relatively stable during the study. The animals dosed at 0.03 mg/kg showed no abnormalities throughout the study. The animals dosed at 0.1 mg/kg showed decreased activity and discolored urine (red) after administration, but no abnormalities on days 2, 3, or 4. The animals dosed at 0.3 mg/kg showed decreased activity and discolored urine (red) after administration, but no abnormalities on days 2, 3, or 4. The animals dosed at 1.0 mg/kg died shortly after administration. There were no abnormalities detected in the control group, e.g., the vehicle dosed group.

### Example R

### Preformulation Studies

Formulations of salinomycin (57 total) were screened using single and/or combinations of solvents and surfactants to determine the suitability of vehicle systems to achieve optimal solubility of salinomycin sodium. Initial screening studies performed identified the composition as listed in Table AA below.

TABLE AA
Preformulation Composition

| Sr. No | Ingredients | Concentration (mg/mL) |
|---|---|---|
| 1 | Salinomycin sodium | 10 |
| 2 | Polyoxyl 15 Hydroxystearate | 50 |
| | (Solutol HS 15) | |
| 3 | Propylene glycol | 40 |
| 4 | Ethanol | 10 |
| 5 | Water For Injection | q.s to 1 mL |

### Example S

### Evaluation, Dilution and I.V Infusion Solution Compatibility Study of Existing I.V Formulation

Formulation development involved evaluation of the composition provided in Example R and optimization to achieve critical attributes related to intravenous administration and also to obtain a physically and chemically stable formulation. Critical attributes for the product evaluated were: clarity, e.g., visual observation, stability, e.g. potency and impurities, pH, e.g., ease of processability, and admixture compatibility.

A trial was undertaken to evaluate feasibility of the salinomycin composition at different batch scales, i.e., 100 mL (Batch A) and 500 mL (Batch B).

### Batch A Method:

Batch A was manufactured at a scale of 100 mL. Approximately 1.12 g Salinomycin sodium was weighed in a beaker. Propylene glycol (approximately 4.0 g) was further weighed and added to beaker containing the API. Solutol HS 15 (available as a white paste at room temperature) was heated at 30° C. to obtain a liquid. Liquified Solutol HS 15 (approximately 5.0 g) was further weighed and added to the dispersion containing salinomycin sodium and propylene glycol. The resultant dispersion obtained was sonicated for 30 mins. to obtain clear yellowish solution taking care not to exceed a temperature above 30° C. Ethanol (approximately 1.0 g) was further weighed and added to the clear yellowish solution under stirring using an overhead stirrer. The volume was adjusted to 100 mL. The clear solution was obtained with a yellowish tinge. This solution was further sonicated for 10 mins. The solution was subsequently filtered through a 0.22 micron PVDF membrane filter. The pH recorded was 7.45 at 22.8° C. Samples were subjected to analysis after filtration.

### Batch B Method:

Batch B was manufactured at a scale of 500 mL. Approximately, 5.60 g of salinomycin sodium was weighed in a beaker. Propylene glycol (approximately 20.0 g) was further weighed and added to a beaker containing API. Solutol HS 15 (available as a white paste at room temperature) was heated at 30° C. to obtain a liquid. Liquified Solutol HS15 (approximately 25.0 g) was further weighed and added to the dispersion containing salinomycin sodium and propylene glycol. The resultant dispersion obtained was sonicated for 90 mins. to obtain a clear yellowish solution taking care not to exceed a temperature above 30° C. Ethanol (approximately 5.0 g) was further weighed and added to the clear yellowish solution under stirring using an overhead stirrer. The volume was adjusted to 500 mL. The clear solution was obtained with a yellowish tinge. This solution was further sonicated for 10 mins. The solution was subsequently filtered through a 0.22 micron PVDF membrane filter. The pH recorded was 7.45 at 23.5° C. Samples were subjected to analysis before and after filtration.

Both the formulations obtained from the Batch A method and the Batch B method were subjected for stress stability studies at 50° C. for 1 week. Results are tabulated in Tables BB and CC below.

TABLE BB
Stress Stability of Salinomycin Sodium Injection
Batch A at 50° C. after 1 week

| | | | Related Substances | | | | | | | | | | |
|---|---|---|---|---|---|---|---|---|---|---|---|---|---|
| Batch A | | | API RELATED IMPURITIES | | | | | UNKNOWN IMPURITIES | | | | | |
| Condition | Appearance | Assay | 0.05 | 0.89 | 1.03 | 1.07 | 1.17 | 0.07 | 0.94 | 0.97 | 1.04 | Total.IMP | pH |
| Initial | Clear | 99.2 | 0.78 | 2.60 | 1.01 | 1.12 | 5.64 | ND | ND | 2.02 | ND | 13.18 | 7.45 |
| | colorless | | | | | | | | | | | | |
| | solution | | | | | | | | | | | | |
| 500 C., | Precipitate in | 95.3 | 2.06 | 2.61 | 0.91 | 1.02 | 5.13 | 2.47 | 1.21 | 2.30 | ND | 17.71 | 7.31 |
| 1 Week | form of | | | | | | | | | | | | |
| | crystals | | | | | | | | | | | | |

TABLE CC
Stress Stability of Salinomycin Sodium Injection
Batch B at 50° C. after 1 week

| | | | Related Substances | | | | | | | | | | |
|---|---|---|---|---|---|---|---|---|---|---|---|---|---|
| Batch B | | Assay | API RELATED IMPURITIES | | | | | UNKNOWN IMPURITIES | | | | | |
| Condition | Appearance | (%) | 0.05 | 0.89 | 1.03 | 1.07 | 1.17 | 0.07 | 0.94 | 0.97 | 1.04 | Total | pH |
| Initial | Clear | 99.2 | 1.44 | 2.65 | 1.03 | 1.11 | 5.74 | ND | ND | 1.77 | ND | 13.74 | 7.45 |
| | colorless | | | | | | | | | | | | |
| | solution | | | | | | | | | | | | |
| Initial | Clear | 100.5 | 0.91 | 2.69 | 1.00 | 1.08 | 5.42 | ND | ND | 1.59 | 0.54 | 13.24 | 7.45 |
| | colorless | | | | | | | | | | | | |
| | solution | | | | | | | | | | | | |

| | | | |
|---|---|---|---|
| 500 C. 1 | Precipitate in | Not analyzed | 7.33 |
| | form of | | |
| | crystals | | |

The compositions provided (Batch A and Batch B) for salinomycin sodium injection yielded a clear colorless solution with an approximate pH of 7.45. Precipitation in the form of crystals were observed in both Batch A and Batch B after exposure to 50° C. after 1 week, and also in the control samples that were stored at 2-8° C. The pH and assay of the formulation was found to remain comparable at 50° C. after 1 week with an increase in unknown impurities. No mass balance was seen in the formulation for assay and related substances.

### Example T

### Investigation of the Precipitate Obtained from Formulation after Exposure to 50° C. For 1 Week by X-Ray Powder Diffraction (XRPD) Studies

### Method:

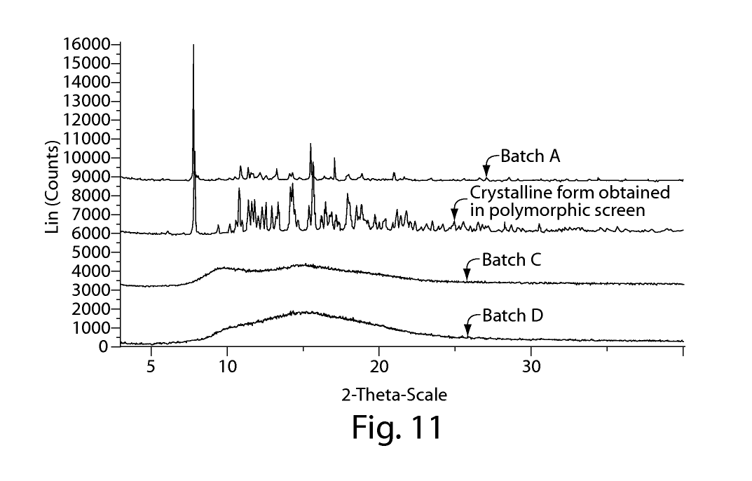

The precipitate observed in Batch A in Example S was isolated and subjected to XRPD analysis. Comparative evaluation was done as shown in FIG. 11 using (1) API used in the formulation (Batch C, manufactured by Neuland Laboratories Ltd., India) and (2) API from a different lot (Batch D). The precipitate obtained from the formulation of Batch A after exposure to 50° C. for 1 week was crystalline in nature and identical to the crystalline form obtained during polymorphic screening.

The Active Pharmaceutical Ingredient (API) used in the formulation along with API of a different lot exhibited amorphous nature. The precipitate obtained in the formulation after exposing to stress stability was crystalline in nature with an identical pattern to the crystalline form obtained during polymorphic screening. The results indicate that the salinomycin sodium has inadequate solubility and form conversion may take place in aqueous solution at increased temperature.

The modification of the co-solvent and the evaluation of the current composition with reduction in strength of API from 10 mg/mL to 5 mg/mL was considered essential.

### Example U

### Stability Studies with Change in Strength

The evaluation of the current composition with an API strength of 5 mg/mL was undertaken. A slight modification of the co-solvent concentration for the strength of 5 mg/mL was evaluated at the same time.

### Method:

Three formulations with salinomycin sodium, at a concentration of 5 mg/mL were manufactured for stability evaluation. The manufacturing process followed was similar to that used for initial feasibility batches. The compositions of the formulations are listed in Table DD. Each formulation was filled in USP, Type I vials with a fill volume of 10 mL. All formulations were subjected to stability studies at 50° C. for 2 weeks, 25° C./40% RH and 2-8° C. for 1, 2 and 3 months. The data was compiled in Tables EE, FF, and GG.

TABLE DD
Different Formulations with Salinomycin Sodium 5 mg/mL

| | Composition | | | | |
|---|---|---|---|---|---|
| | Salinomycin | Solutol | Propylene | Ethanol | |
| | sodium | HS15 | glycol | (mg/ | |
| Batch | (mg/mL) | (mg/mL) | (mg/mL) | mL) | Water |
| E | 5 | 50 | 40 | 10 | quantity sufficient |
| | | | | | to 1 mL |
| F | 5 | 50 | 80 | 20 | quantity sufficient |
| | | | | | to 1 mL |
| G | 5 | 50 | 40 | 50 | quantity sufficient |
| | | | | | to 1 mL |

TABLE EE
Stability Data for Salinomycin Dodium Injection 5 mg/mL, Batch E

| | | | Related Substances | | | | | | | |
|---|---|---|---|---|---|---|---|---|---|---|
| | | Assay | API RELATED IMPURITIES | | | | | | | |
| Condition | Appearance | %) | 0.89 | 1.03 | 1.04 | 1.05 | 1.06 | 1.07 | 1.17 | 1.38 |
| Initial | Slight hazy | 98.5 | ND | 1.72 | 1.56 | 1.21 | ND | ND | 5.45 | ND |
| | under High | | | | | | | | | |
| | intensity | | | | | | | | | |
| | light | | | | | | | | | |
| 500 C. | Slight hazy | 96.1 | 0.94 | 1.72 | 1.13 | ND | ND | 1.10 | 5.56 | ND |
| 1 Week | under High | | | | | | | | | |
| | intensity | | | | | | | | | |
| | light | | | | | | | | | |
| 500 C. | Slight hazy | 86.3 | 0.95 | 1.25 | 1.31 | ND | ND | 1.06 | 5.08 | ND |
| 2 Week | under High | | | | | | | | | |
| | intensity | | | | | | | | | |
| | light | | | | | | | | | |
| 25° C./60% | Swirling | 97.2 | 1.26 | 1.88 | 1.28 | ND | ND | 1.21 | 5.26 | ND |
| RH 1 M | effect | | | | | | | | | |
| 25° C./60% | Swirling | 93.8 | 1.48 | 1.29 | 1.14 | ND | ND | 5.03 | ND | ND |
| RH 2 M | effect | | | | | | | | | |
| 25° C./60% | Swirling | 88.1 | 2.59 | 1.77 | 0.96 | 0.10 | 1.24 | ND | 4.51 | 3.45 |
| RH 3 M | effect | | | | | | | | | |
| 2-8° C. 1 M | Swirling | 97.8 | 1.44 | 1.75 | 0.99 | ND | ND | 1.14 | 5.81 | ND |
| | effect | | | | | | | | | |
| 2-8° C. 2 M | Swirling | 98.8 | 1.37 | 0.74 | 1.27 | ND | ND | 5.41 | ND | ND |
| | effect | | | | | | | | | |
| 2-8° C. 3 M | Swirling | 96.0 | 2.45 | 1.66 | 0.68 | 0.12 | 1.03 | ND | 5.21 | ND |
| | effect | | | | | | | | | |
| | | Related Substances | | | | | | | | |
| | | UNKNOWN IMPURITIES | | | | | | | Total. | |
| Condition | Appearance | 0.91 | 0.93 | 0.94 | 0.95 | 0.96 | 0.97 | 1.46 | IMP | pH |
| Initial | Slight hazy | ND | ND | ND | ND | ND | ND | ND | 9.95 | 7.33 |
| | under High | | | | | | | | | |
| | intensity | | | | | | | | | |
| | light | | | | | | | | | |
| 500 C. | Slight hazy | 6.03 | ND | 1.35 | ND | 1.63 | ND | ND | 19.47 | 7.31 |
| 1 Week | under High | | | | | | | | | |
| | intensity | | | | | | | | | |
| | light | | | | | | | | | |
| 500 C. | Slight hazy | 11.47 | ND | ND | 2.56 | ND | 3.84 | ND | 27.52 | 7.05 |
| 2 Week | under High | | | | | | | | | |
| | intensity | | | | | | | | | |
| | light | | | | | | | | | |
| 25° C./60% | Swirling | 2.96 | ND | 0.29 | ND | ND | 2.12 | ND | 16.25 | 7.15 |
| RH 1 M | effect | | | | | | | | | |
| 25° C./60% | Swirling | 8.64 | 0.80 | 2.77 | ND | ND | 1.65 | ND | 22.79 | 7.18 |
| RH 2 M | effect | | | | | | | | | |
| 25° C./60% | Swirling | 11.42 | ND | 0.93 | ND | 0.68 | 5.02 | 0.73 | 33.45 | 7.32 |
| RH 3 M | effect | | | | | | | | | |
| 2-8° C. 1 M | Swirling | 0.82 | ND | ND | ND | ND | 2.35 | ND | 14.30 | 7.43 |
| | effect | | | | | | | | | |
| 2-8° C. 2 M | Swirling | 2.7 | 0.75 | 2.74 | ND | 2.91 | 1.74 | ND | 16.72 | 7.43 |
| | effect | | | | | | | | | |
| 2-8° C. 3 M | Swirling | 2.39 | ND | 0.38 | ND | ND | 2.98 | 0.93 | 17.84 | 7.34 |
| | effect | | | | | | | | | |

TABLE FF
Stability Data for Salinomycin Sodium Injection 5 mg/mL (Batch F)

| | | | Related Substances | | | | | | | |
|---|---|---|---|---|---|---|---|---|---|---|
| | | Assay | API RELATED IMPURITIES | | | | | | | |
| Condition | Appearance | (%) | 0.89 | 1.03 | 1.04 | 1.05 | 1.06 | 1.07 | 1.17 | 1.38 |
| Initial | Slight hazy | 97.9 | ND | 1.72 | 1.56 | 1.23 | ND | ND | 5.40 | ND |
| | under High | | | | | | | | | |
| | intensity light | | | | | | | | | |
| 500 C. | Slight hazy | 96.4 | 1.67 | 1.70 | 0.93 | ND | ND | 1.16 | 5.46 | ND |
| 1 Week | under High | | | | | | | | | |
| | intensity light | | | | | | | | | |
| 500 C. | Slight hazy | 85.5 | 0.99 | 1.25 | 1.30 | ND | ND | 1.03 | 4.99 | ND |
| 2 Week | under High | | | | | | | | | |
| | intensity light | | | | | | | | | |
| 25° C./60% RH | Swirling effect | 97.6 | 1.59 | 1.90 | 1.12 | ND | ND | 1.10 | 5.58 | ND |
| 1 M | | | | | | | | | | |
| 25° C./60% RH | Swirling effect | 94.8 | 1.66 | 0.74 | 1.13 | ND | ND | 5.29 | ND | ND |
| 2 M | | | | | | | | | | |
| 25° C./60% RH | Swirling effect | 91.2 | 3.10 | 1.83 | 1.13 | 0.19 | 1.33 | ND | 5.06 | ND |
| 3 M | | | | | | | | | | |
| 2-8° C. 1 M | Swirling effect | 99.6 | 1.45 | 1.74 | 0.92 | ND | ND | 0.93 | 5.24 | ND |
| 2-8° C. 2 M | Swirling effect | 99.8 | 1.71 | 0.61 | 0.94 | ND | ND | 5.55 | ND | ND |
| 2-8° C. 3 M | Swirling effect | 97.2 | 3.08 | 1.90 | 1.01 | 0.28 | 0.93 | ND | 5.10 | ND |
| | | Related Substances | | | | | | | | |
| | | UNKNOWN IMPURITIES | | | | | | | Total. | |
| Condition | Appearance | 0.91 | 0.93 | 0.94 | 0.95 | 0.96 | 0.97 | 1.46 | IMP | pH |
| Initial | Slight hazy | ND | ND | ND | ND | ND | ND | ND | 9.92 | 7.51 |
| | under High | | | | | | | | | |
| | intensity light | | | | | | | | | |
| 500 C. | Slight hazy | 6.39 | ND | 1.60 | ND | 1.76 | ND | ND | 20.66 | 7.37 |
| 1 Week | under High | | | | | | | | | |
| | intensity light | | | | | | | | | |
| 500 C. | Slight hazy | 13.55 | ND | ND | 2.32 | ND | 4.32 | ND | 29.74 | 7.15 |
| 2 Week | under High | | | | | | | | | |
| | intensity light | | | | | | | | | |
| 25° C./60% RH | Swirling effect | 2.66 | ND | 0.35 | ND | ND | 2.42 | ND | 16.73 | 7.37 |
| 1 M | | | | | | | | | | |
| 25° C./60% RH | Swirling effect | 7.21 | 0.73 | 2.37 | ND | ND | 1.44 | ND | 20.58 | 7.44 |
| 2 M | | | | | | | | | | |
| 25° C./60% RH | Swirling effect | 9.40 | ND | 1.19 | ND | 3.41 | ND | 0.34 | 26.99 | 7.33 |
| 3 M | | | | | | | | | | |
| 2-8° C. 1 M | Swirling effect | 0.81 | ND | ND | ND | ND | 2.40 | ND | 13.50 | 7.44 |
| 2-8° C. 2 M | Swirling effect | 2.33 | 0.62 | 2.29 | ND | ND | 1.78 | ND | 15.83 | 7.54 |
| 2-8° C. 3 M | Swirling effect | 2.19 | ND | 0.39 | ND | ND | 2.93 | 0.87 | 18.68 | 7.30 |

TABLE GG
Stability Data for Salinomycin Sodium Injection 5 mg/mL (Batch G)

| | | | Related Substances | | | | | | | |
|---|---|---|---|---|---|---|---|---|---|---|
| | | Assay | API RELATED IMPURITIES | | | | | | | |
| Condition | Appearance | (%) | 0.89 | 1.03 | 1.04 | 1.05 | 1.06 | 1.07 | 1.17 | 1.38 |
| Initial | Slight hazy | 97.4 | ND | 1.73 | 1.54 | 1.22 | ND | ND | 5.50 | ND |
| | under High | | | | | | | | | |
| | intensity light | | | | | | | | | |
| 500 C. | Slight hazy | 96.1 | 1.75 | 1.69 | 0.74 | ND | ND | 1.05 | 5.45 | ND |
| 1 Week | under High | | | | | | | | | |
| | intensity light | | | | | | | | | |
| 500 C. | Slight hazy | 86.0 | 1.02 | 1.23 | 1.34 | ND | ND | 1.05 | 4.98 | ND |
| 2 Week | under High | | | | | | | | | |
| | intensity light | | | | | | | | | |
| 25° C./60% | Swirling effect | 96.2 | 1.42 | 1.77 | 1.04 | ND | ND | 1.09 | 5.27 | ND |
| RH 1 M | | | | | | | | | | |
| 25° C./60% | Swirling effect | 95.7 | 1.89 | 0.54 | 1.01 | ND | ND | 5.75 | ND | ND |
| RH 2 M | | | | | | | | | | |
| 25° C./60% | Swirling effect | 91.6 | 3.48 | 1.73 | 0.68 | 0.08 | 1.07 | ND | 4.90 | ND |
| RH 3 M | | | | | | | | | | |
| 2-8° C. 1 M | Swirling effect | 98.3 | 1.48 | 1.68 | 0.96 | ND | ND | 1.01 | 5.39 | ND |
| 2-8° C. 2 M | Swirling effect | 99.6 | 1.76 | 0.74 | 0.93 | ND | ND | 5.38 | ND | ND |
| 2-8° C. 3 M | Swirling effect | 97.8 | 3.17 | 1.72 | 0.89 | 0.11 | 0.94 | ND | 5.27 | ND |

| | | Related Substances | | | | | | | | |
|---|---|---|---|---|---|---|---|---|---|---|
| | | UNKNOWN IMPURITIES | | | | | | | Total | |
| Condition | Appearance | 0.91 | 0.93 | 0.94 | 0.95 | 0.96 | 0.97 | 1.46 | IMP | pH |
| Initial | Slight hazy | ND | ND | ND | ND | ND | ND | ND | 9.9810 | 7.41 |
| | under High | | | | | | | | | |
| | intensity light | | | | | | | | | |
| 500 C. | Slight hazy | 6.30 | ND | 2.46 | ND | 1.99 | ND | ND | 21.4311 | 7.38 |
| 1 Week | under High | | | | | | | | | |
| | intensity light | | | | | | | | | |
| 500 C. | Slight hazy | 14.40 | ND | ND | 2.37 | ND | 4.59 | ND | 30.9812 | 7.24 |
| 2 Week | under High | | | | | | | | | |
| | intensity light | | | | | | | | | |
| 25° C./60% | Swirling effect | 2.83 | ND | 0.32 | ND | ND | 2.54 | ND | 16.2814 | 7.42 |
| RH 1 M | | | | | | | | | | |
| 25° C./60% | Swirling effect | 7.63 | 0.63 | 1.25 | ND | ND | 1.44 | ND | 20.1515 | 7.43 |
| RH 2 M | | | | | | | | | | |
| 25° C./60% | Swirling effect | 9.77 | ND | 1.11 | ND | ND | 3.56 | 0.46 | 26.8517 | 7.30 |
| RH 3 M | | | | | | | | | | |
| 2-8° C. 1 M | Swirling effect | 0.82 | ND | ND | ND | ND | 2.52 | ND | 13.8614 | 7.47 |
| 2-8° C. 2 M | Swirling effect | 2.50 | 0.61 | 2.37 | ND | ND | 1.77 | ND | 16.0615 | 7.53 |
| 2-8° C. 3 M | Swirling effect | 2.28 | ND | 0.33 | ND | ND | 2.82 | 0.55 | 18.0812 | 7.38 |

All formulations were observed visually as clear colorless solutions. No precipitate was observed. Stability samples were stored at 2-8° C. before analysis. The appearance of these solutions when observed immediately on removal from the refrigerator, showed haziness which disappeared on thawing the samples at room temperature. This was interpreted as an impact of Solutol HS15 used in the formulation. The pH was found to remain stable for all formulations at 25° C./60% RH and 2-8° C. A significant drop was seen in assay at 25° C./60% RH for all formulations. A minimal drop was seen in assay at 2-8° C. for formulation Batch E with no change in assay for Batch F and Batch G. A similar trend of impurities was observed in all formulations. No mass balance was seen for Assay and Related substances. A significant increase in impurities was found in all formulations with a major contribution by unknown impurities at retention times 0.91, 0.94, 0.96 and 1.46 at 25° C./60% RH.

All the formulations were found to be stable at 2-8° C. for Assay and pH. The trend of the data was found to be similar in all formulations. It was interpreted that the existing analytical method is not stability indicating since no mass balance between assay and related substances was observed. Impurities observed at retention times 0.91, 0.94, 0.96 and 1.46 needed identification. The compositions provided in Example R of the Salinomycin sodium at a concentration of 5 mg/mL was therefore subjected to admixture studies.

### Example V

### Admixture Studies

Admixture studies were conducted to evaluate the compatibility of salinomycin sodium injection 5 mg/mL with various diluents. Diluents evaluated were normal saline, 5% dextrose injection, and 5% Dextrose injection in saline.

### Batch H Method:

Batch H was manufactured at a scale of 500 mL. Approximately 2.80 g of Salinomycin sodium was weighed in a beaker. Propylene glycol (approximately 20.0 g) was further weighed and added to a beaker containing the API. Solutol HS15 (available as a white paste at room temperature) was heated at 30° C. to obtain a liquid. Liquified Solutol HS15 (approximately 25.0 g) was further weighed and added to the dispersion containing salinomycin sodium and propylene glycol. The resultant dispersion obtained was sonicated for 30 mins. to obtain a clear yellowish solution. Ethanol (approximately 5.0 g) was further weighed and added to the clear yellowish solution under stirring conditions using an overhead stirrer. The volume was adjusted to 500 mL. The clear solution was obtained with a yellowish tinge. This solution was further sonicated for 10 mins. The solution was further filtered through a 0.22 micron PVDF membrane filter. The pH recorded was 7.15 at 26.0° C. Samples were subjected to analysis after filtration. The results of Batch H are tabulated in Table HH. Each 10 mL of the formulation was added to 500 mL of diluents. Normal saline, 5% dextrose injection, and 5% dextrose injection in saline were added, respectively, to obtain the concentration of approximately 0.098 mg/mL. Samples were analyzed at 0 hours, 6 hours and 24 hours at room temperature and 2-8° C. for visual appearance, assay and pH as listed in Tables II, JJ, and KK. Diluents were procured from Claris Lifesciences, IP grade with specifications equivalent to USP.

TABLE HH
Results from Batch H

| | | | Related Substances | | | | | | | | | | |
|---|---|---|---|---|---|---|---|---|---|---|---|---|---|
| Batch H | | | API RELATED IMPURITIES | | | | | UNKNOWN IMPURITIES | | | | | |
| Condition | Assay | 0.05 | 0.89 | 1.03 | 1.04 | 1.07 | 1.17 | 0.07 | 0.94 | 0.97 | 1.04 | Total. IMP | pH |
| Initial | 98.1 | ND | 6.92 | 1.74 | 0.87 | 0.97 | 5.28 | ND | ND | ND | ND | 15.78 | 7.56 |

TABLE II
Admixture Studies in Normal Saline
Normal Saline

| | | Acceptance | Initial | | 6 Hr | | 24 Hr | |
|---|---|---|---|---|---|---|---|---|
| Sr. No. | Parameter | Criteria | R.T | 2-8° C. | R.T | 2-8° C. | R.T | 2-8° C. |
| 1. | Appearance | Clear colorless | Clear colorless | Clear colorless | Clear | Clear | Clear | Clear |
| | | solution free from | solution | solution free | colorless | colorless | colorless | colorless |
| | | visible particles | free from visible | from visible | solution | solution free | solution free | solution free |
| | | | particles | particles | free from | from visible | from visible | from visible |
| 2. | Assay | Not less than | 93.7% | 93.2% | 92.8% | 93.8% | 92.8% | 92.5% |
| | | 90% and Not | | | | | | |
| | | more than | | | | | | |
| 3. | Apparent pH | N/A | 6.05 | 6.07 | 5.90 | 5.85 | 5.86 | 5.76 |

TABLE JJ
Admixture Studies in 5% Dextrose Injection
Normal Saline

| | | Acceptance | Initial | | 6 Hr | | 24 Hr | |
|---|---|---|---|---|---|---|---|---|
| Sr. No. | Parameter | Criteria | R.T | 2-8° C. | R.T | 2-8° C. | R.T | 2-8° C. |
| 1. | Appearance | Clear colorless | Clear colorless | Clear | Clear | Clear | Clear | Clear |
| | | solution free from | solution | colorless | colorless | colorless | colorless | colorless |
| | | visible particles | free from | solution free | solution free | solution free | solution free | solution free |
| | | | visible | from visible | from visible | from visible | from visible | from visible |
| 2. | Assay | Not less than | 96.5% | 96.7% | 95.8% | 96.7% | 89.2% | 96.3% |
| | | 90% and Not | | | | | | |
| | | more than | | | | | | |
| 3. | Apparent pH | N/A | 4.50 | 4.48 | 4.42 | 4.52 | 4.32 | 4.44 |

TABLE KK
Admixture Studies in 5% Dextrose in Normal Saline
Normal Saline

| | | Acceptance | Initial | | 6 Hr | | 24 Hr | |
|---|---|---|---|---|---|---|---|---|
| Sr. No. | Parameter | Criteria | R.T | 2-8° C. | R.T | 2-8° C. | R.T | 2-8° C. |
| 1. | Appearance | Clear colorless | Clear colorless | Clear | Clear | Clear | Clear | Clear |
| | | solution free from | solution | colorless | colorless | colorless | colorless | colorless |
| | | visible particles | free from | solution free | solution free | solution free | solution free | solution free |
| | | | visible | from visible | from visible | from visible | from visible | from visible |
| 2. | Assay | Not less than | 100.1% | 100.8% | 93.7% | 93.0% | 91.1% | 90.3% |
| | | 90% and Not | | | | | | |
| | | more than | | | | | | |
| 3. | Apparent pH | N/A | 4.48 | 5.18 | 4.40 | 5.12 | 4.38 | 5.08 |

The formulation was found to remain stable in normal saline up to 24 hours at room temperature. The formulation was also found to remain stable in 5% dextrose up to 6 hours at room temperature. The formulation with 5% dextrose in saline was found unstable at 6 hours. Therefore, normal saline was deemed compatible for the preparation of the admixture.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. An aqueous composition comprising salinomycin or a pharmaceutically-acceptable salt thereof, a miscible organic solvent, and an emulsifying agent.

2. The aqueous composition of claim 1, wherein the aqueous composition comprises 0.05 mg/mL to 50 mg/mL salinomycin or a pharmaceutically-acceptable salt thereof.

3. The aqueous composition of claim 1, wherein the aqueous composition comprises 1% to 40% miscible organic solvent (v/v).

4. The aqueous composition of claim 1, wherein the aqueous composition comprises 0.1% to 30% emulsifying agent (v/v).

5. The aqueous composition of claim 1, wherein the miscible organic solvent is ethanol, propylene glycol, or DMSO.

6. The aqueous composition of claim 5, wherein the miscible organic solvent comprises ethanol and propylene glycol.

7. The aqueous composition of any claim 1, wherein the emulsifying agent is Tween® 80, Solutol® HS 15, or Cremophor EL.

8. The aqueous composition of claim 7, wherein the emulsifying agent is Solutol® HS 15.

9. The aqueous composition of claim 1, additionally comprising a preservative.

10. A dosage form comprising an aqueous composition of claim 1.

11. A kit comprising an aqueous composition or dosage form of claim 1.

12. The kit of claim 11, additionally comprising instructions for the administration of the aqueous composition or dosage form to a subject.

13. An aqueous composition comprising 0.01-15 mg/mL salinomycin or a pharmaceutically-acceptable salt thereof, 0.5-5% (v/v) ethanol, 1-10% (v/v) propylene glycol, and 1-20% (v/v) Solutol® HS 15.

14. An aqueous composition comprising water, salinomycin or a pharmaceutically-acceptable salt thereof, a miscible organic solvent, and an emulsifying agent for delivering salinomycin to a subject at 0.5 to 5 mg/kg intravenously.

15. The aqueous composition of claim 14, wherein the aqueous composition comprises 0.5-5% (v/v) ethanol, 1-10% (v/v) propylene glycol, and 1-20% (v/v) Solutol® HS 15.

16. A method of treating a subject having been diagnosed with a proliferative disease, comprising administering to the subject an aqueous salinomycin composition, thereby treating the subject.

17. The method of claim 16, wherein the subject is also administered a chemotherapeutic agent.

18. The method of claim 17, wherein the chemotherapeutic agent is an alkylating agent, an antimetabolite, an anthracycline, an alkaloid, a topoisomerase inhibitor, a platinum compound, or a PARP inhibitor.

19. The method of claim 16, wherein the chemotherapeutic agent is administered simultaneously with the aqueous salinomycin composition.

20. The method of claim 16, wherein the chemotherapeutic agent is administered sequentially with the aqueous salinomycin composition.

21. The method of claim 16, wherein the proliferative disease is cancer.

22. The method of claim 16, wherein the proliferative disease is metastatic cancer.

23. The method of claim 16, wherein the proliferative disease is cancer associated with cancer stem cells.

24. The method of claim 16, wherein the proliferative disease is cancer associated with mesenchymal cells.

25. The method of claim 16, wherein the proliferative disease is a colon carcinoma, a pancreatic cancer, a breast cancer, an ovarian cancer, a prostate cancer, a squamous cell carcinoma, a cervical cancer, a lung carcinoma, a small cell lung carcinoma, a bladder carcinoma, a basal cell carcinoma, an adenocarcinoma, a sweat gland carcinoma, a sebaceous gland carcinoma, a papillary carcinoma, a papillary adenocarcinoma, a cystadenocarcinoma, a medullary carcinoma, a bronchogenic carcinoma, a renal cell carcinoma, a hepatocellular carcinoma, a bile duct carcinoma, a choriocarcinoma, a seminoma, an embryonal carcinoma, a Wilms' tumor, or a testicular tumor.

26. The method of claim 25, wherein the proliferative disease is breast cancer.

27. The method of claim 26, wherein the breast cancer does not express genes for estrogen receptors, progesterone receptors, or Her2/neu receptors.

28. The method of claim 16, wherein the aqueous salinomycin composition additionally comprises a miscible organic solvent.

29. The method of claim 28, wherein the miscible organic solvent is ethanol, propylene glycol, or DMSO.

30. The method of claim 29, wherein the miscible organic solvent comprises ethanol and propylene glycol.

31. The method of claim 16, wherein the aqueous salinomycin composition additionally comprises a solubilizer, a surfactant, an emulsifying agent, or a stabilizer.

32. The method of claim 31, wherein the emulsifying agent is Tween® 80, Solutol® HS 15, or Cremophor EL.

33. The method of claim 32, wherein the emulsifying agent is Solutol® HS 15.

34. The method of claim 16, wherein the aqueous salinomycin composition additionally comprises a preservative.

35. The method of claim 16, wherein the aqueous salinomycin composition comprises 1%, e.g. to 40% miscible organic solvent (v/v).

36. The method of claim 16, wherein the aqueous salinomycin composition comprises 0.1% to 30% emulsifying agent (v/v).

37. The method of claim 16, wherein the aqueous salinomycin composition comprises 0.05 mg/ml to 50 mg/ml salinomycin or a pharmaceutically-acceptable salt thereof.

38. The method of claim 16, wherein the aqueous salinomycin composition is administered at a dose of 0.001 to 10 mg/kg.

39. The method of claim 16, wherein the aqueous salinomycin composition is administered to the subject intravenously or subcutaneously.

40. The method of claim 39, wherein the aqueous salinomycin composition is administered over more than 30 minutes.

41. The method of claim 39, wherein the aqueous salinomycin composition is administered every day, every other day, three times a week, twice weekly, weekly, every other week, every third week, every fourth week, or monthly.

42. A method of treating a subject having a disorder in which a cancer stem cell biomarker has been detected, the method comprising administering to the subject an aqueous salinomycin composition, thereby treating the subject.

43. A method of treating a subject having a disorder in which a Wnt pathway activation biomarker has been detected, the method comprising administering to the subject an aqueous salinomycin composition, thereby treating the subject.

44. The method of claim 42 wherein the stem cell biomarker is selected from E-cadherin, TWIST expression, and a CD44/CD24 cell surface marker profile.

45. The method of claim 42, wherein the method further comprises obtaining a cell or tissue sample from the subject.

46. The method of claim 44, wherein the E-cadherin and/or TWIST expression in the cancer is determining by measuring a level of E-cadherin and/or TWIST protein and/or RNA expression in the cancer, and optionally comparing the level to a reference standard.

47. The method of claim 46, wherein the reference standard is the level of E-cadherin and/or TWIST protein and/or RNA expression in a cancer stem cell.

48. The method of claim 46, wherein the reference standard is the level of E-cadherin and/or TWIST protein and/or RNA expression in a cancer cell that is not a cancer stem cell.

49. The method of claim 42 wherein the stem cell biomarker is selected from CD20, CD24, CD34, CD38, CD44, CD45, CD105, CD133, CD166, EpCAM, ESA, SCA1, Pecam, and Stro1.

50. The method of claim 42, wherein the cancer is a colon carcinoma, a pancreatic cancer, a breast cancer, an ovarian cancer, a prostate cancer, a squamous cell carcinoma, a cervical cancer, a lung carcinoma, a small cell lung carcinoma, a bladder carcinoma, a squamous cell carcinoma, a basal cell carcinoma, an adenocarcinoma, a sweat gland carcinoma, a sebaceous gland carcinoma, a papillary carcinoma, a papillary adenocarcinoma, a cystadenocarcinoma, a medullary carcinoma, a bronchogenic carcinoma, a renal cell carcinoma, a hepatocellular carcinoma, a bile duct carcinoma, a choriocarcinoma, a seminoma, a embryonal carcinoma, a Wilms' tumor, or a testicular tumor.

51. A method of making an aqueous salinomycin composition, comprising:
contacting a pharmaceutically acceptable salinomycin salt with a miscible organic solvent to make a solution;
contacting the solution with a solubilizer and/or emulsifying agent; and
contacting the solution with water,
to thereby make an aqueous salinomycin composition.

52. The use of an aqueous salinomycin composition for the treatment of a proliferative disease in a subject in need thereof.

53. A method of inhibiting the growth or differentiation of a cancer stem cell, comprising contacting the cancer stem cell with an aqueous salinomycin composition.

54. The method of claim 53, wherein the cancer stem cell has activity in a transcription factor selected from Snail1, Snail2, Goosecoid, FoxC1, FoxC2, TWIST, E2A, SIP-1/Zeb-2, dEF1/Zeb1, LEF, Myc, HMGA2, TAZ, Klf8, HIF-1, HOXB7, SIM2s, and Fos.

55. The method of claim 53, wherein the aqueous salinomycin composition comprises 0.01-15 mg/mL salinomycin, 0.5-5% (v/v) ethanol, 1-10% (v/v) propylene glycol, and 1-20% (v/v) Solutol® HS 15.

56. A method of inhibiting the growth or differentiation of a mesenchymal cell, a cancer stem cell, a tumor-initiating cell, a mesenchymal-like cell associated with cancer, or a mesenchymal cancerous cell, comprising contacting the mesenchymal cell, the cancer stem cell, the tumor-initiating cell, the mesenchymal-like cell associated with cancer, or the mesenchymal cancerous cell with an aqueous salinomycin composition.

57. The method of claim 56, wherein the mesenchymal cell has activity in a transcription factor selected from Snail1, Snail2, Goosecoid, FoxC1, FoxC2, TWIST, E2A, SIP-1/Zeb-2, dEF1/Zeb1, LEF, Myc, HMGA2, TAZ, Klf8, HIF-1, HOXB7, SIM2s, and Fos.

58. The method of claim 56, wherein the aqueous salinomycin composition comprises 0.01-15 mg/mL salinomycin, 0.5-5% (v/v) ethanol, 1-10% (v/v) propylene glycol, and 1-20% (v/v) Solutol® HS 15.

59. The aqueous composition of claim 1, wherein the aqueous composition comprises 5 mg/mL salinomycin or a pharmaceutically-acceptable salt thereof.

60. An aqueous composition comprising 5 mg/mL salinomycin or a pharmaceutically-acceptable salt thereof, a miscible organic solvent, and an emulsifying agent.

61. The aqueous composition of claim 1, wherein the aqueous composition comprises 0.1 mg/mL salinomycin or a pharmaceutically-acceptable salt thereof.

62. The aqueous composition of claim 1, wherein the aqueous composition comprises 0.5 mg/mL salinomycin or a pharmaceutically-acceptable salt thereof.
